(19) United States
(12) Patent Application Publication
(10) Pub. No.: US 2020/0172503 A1
(43) Pub. Date: Jun. 4, 2020

(54) PROCESS FOR PURIFYING TETRAHYDROCANNABINOL USING A CHROMATOGRAPHIC STATIONARY PHASE

(71) Applicant: Orochem Technologies Inc., Naperville, IL (US)

(72) Inventors: Anil OROSKAR, Oak Brook, IL (US); Kunal GULATI, Naperville, IL (US); David HOUSE, Arlington Heights, IL (US); Asha OROSKAR, Oak Brook, IL (US); Gautham OROSKAR, Oak Brook, IL (US)

(73) Assignee: Orochem Technologies Inc., Naperville, IL (US)

(21) Appl. No.: 16/700,773

(22) Filed: Dec. 2, 2019

Related U.S. Application Data
(60) Provisional application No. 62/775,222, filed on Dec. 4, 2018.

(51) Int. Cl. C07D 311/80

(52) U.S. Cl. CPC C07D 311/80

(57) ABSTRACT

Embodiments of a method of purifying tetrahydrocannabinol (THC) from a composition containing THC and at least one impurity, e.g., from pesticides, waxes, lipids, pigments, and other cannabinoids, can use a continuous simulated moving bed process, a batch column chromatography method, or a single column, and a combination of one or more of a sequence of purification steps including: filtration, decolorization, activation or decarboxylation, dewaxing, polishing, and crystallization to separate a cannabinoid from the cannabis plant and to provide various cannabinoid products. The THC products can be used in various pharmaceutical and nutraceutical applications.

### CROSS-REFERENCE TO RELATED APPLICATION

This patent application claims the benefit of priority to U.S. Patent Application No. 62/775,222, filed Dec. 4, 2018, and entitled, “Process for Purifying Tetrahydrocannabinol Using a Chromatographic Stationary Phase,” which is incorporated in its entirety herein by this reference.

### BACKGROUND

The present disclosure relates to processes for purifying cannabis and, more particularly, to methods of purifying tetrahydrocannabinol (THC) from a composition containing THC and at least one impurity.

The term cannabis refers to the genus Cannabis, which contains three species Cannabis sativa, Cannabis indica, and Cannabis ruderalis. All three species are of the family Cannabaceae, which also includes the genus Humulus, or hops. Cannabis is a flowering plant that is indigenous to central Asia and India. Humans have been cultivating and using cannabis for thousands of years, going back to the ancient Romans, Greeks, and the Islamic empires of the Middle East and Africa. There are at least 113 different cannabinoids present in the cannabis plant. All of the classes of cannabinoids are derived from a common precursor compound, cannabigerol (CBG). The cannabis plant also contains a variety of terpenoids. Most such compounds are lipophilic and phenolic.

The legalization of medicinal Cannabis is occurring across the United States and in many other countries. As a result, the global demand for cannabinoids is increasing. In addition, a number of recent medical studies report health benefits of many cannabinoids. Cannabis contains over 85 cannabinoids, and most of them have been found to have therapeutically beneficial properties. Examples of such cannabinoids found to have therapeutic properties include tetrahydrocannabinol (THC), cannabidiol (CBD), cannabigerol (CBG) and cannabinol (CBN).

THC oil is a nutraceutical that has growing market place demand since its potential in effectively providing treatment for seizures, nausea, vomiting, lack of appetite, pain, arthritis, inflammation, and other conditions. Cannabinoids, such as THC, can be extracted from cannabis of the three species Cannabis sativa, Cannabis indica, and Cannabis ruderalis using a hydrocarbon solvent such as butane, a supercritical solvent such as carbon dioxide, or ethanol. Butane extraction and supercritical CO₂ extraction, have accounted for the majority of production of cannabinoid concentrates currently available on the market. A third extraction method, based on ethanol has been gaining market share as a solvent of choice for manufacturing high-quality cannabis extracts.

Each of the aforementioned extraction processes can be used to isolate THC. However, said extraction processes generally also result the presence of impurities such as, for example, non-polar waxes/lipids, sugars and carbohydrates, proteins, chlorophyll, color pigments, pesticides, and other cannabinoids. In particular, many cannabis products contain pesticides like bifenazate, spinomesifen, and bifenthrin at levels higher than the maximum allowable amount for edible or smoked product. In order to remove all of the impurities associated with the extraction process, purification methods are necessary to meet high purity specifications. Traditional THC purification methods like molecular distillation result in low THC recovery yields and often can produce inefficient and unreproducible removal of pesticides.

There is a continued need for new methods for recovering and purifying a tetrahydrocannabinol (THC) rich oil. To satisfy the growing demand for tetrahydrocannabinol (THC), there is a need for an efficient extraction process that can be carried out on a commercial scale to produce high purity tetrahydrocannabinol (THC) products.

It will be appreciated that this background description has been created by the inventors to aid the reader and is not to be taken as an indication that any of the indicated problems were themselves appreciated in the art. While the described principles can, in some aspects and embodiments, alleviate the problems inherent in other systems, it will be appreciated that the scope of the protected innovation is defined by the attached claims and not by the ability of any disclosed feature to solve any specific problem noted herein.

### SUMMARY

In one aspect, the present disclosure is directed to embodiments of a method for the purification and separation of THC from cannabis. For example, in embodiments, methods following principles of the present disclosure can be used to separate a desired cannabinoid (i.e., tetrahydrocannabinol), i.e., to increase its purity, from other cannabinoids and impurities such as pesticides.

Thus, in some aspects, a method of separating THC from a cannabis plant (i.e., cannabis biomass) can be used to process a cannabis plant including THC and at least one impurity. In one embodiment, the method includes combining the cannabis plant (e.g., ground, flour, trim, etc.) and a solvent to form a crude cannabis extract stream. The crude cannabis extract stream can be used directly, or can be further processed (e.g., decolorized and/or decarboxylated), to provide the composition from which the THC is to be purified. In some aspects, a method of purifying THC can be used to process crude THC oil including THC and at least one impurity. Crude THC oil can refer to any composition comprising THC. In certain embodiments, the crude THC oil does not include chlorophyll and other pigments. The crude THC oil can be used directly, or can be further processed (e.g., decarboxylated).

In some aspects, a method of purifying tetrahydrocannabinol (THC) from a composition containing THC and at least one impurity is provided. In one embodiment, the method includes preparing a feedstock stream that includes the composition. The feedstock stream is passed through one or more stationary phases to provide an eluate stream. The eluate stream has a higher purity of the THC than in the feedstock stream as measured by weight percentage of the THC content. The one or more stationary phases comprises: (i) a first adsorbent comprising a silica adsorbent having Si—OH groups and an average particle diameter between 60-200 microns, (ii) a second adsorbent comprising a modified hydrophobic adsorbent having an average bulk density of from about 0.4 g/mL to about 0.6 g/mL, the modified hydrophobic adsorbent comprising at least one of a styrene-divinylbenzene (DVB) resin or a poly(methyl methacrylate) (PMMA) resin, (iii) a third adsorbent comprising a modified activated carbon adsorbent having an average particle size range of from about 40 to about 1700 microns, or (iv) any mixture thereof.

In some aspects, a method of purifying tetrahydrocannabinol (THC) from a composition containing THC and at least one impurity is provided. In one embodiment, the method includes preparing a first feedstock stream that includes the composition. The first feedstock stream further comprises a first major solvent of a first polarity. The first feedstock stream is passed through a first stationary phase to provide a first eluate stream. The first eluate stream has a higher purity of the THC than in the first feedstock stream as measured by weight percentage of the THC content. The method further includes removing at least some of the first major solvent of the first polarity from the first eluate stream to produce a reduced first eluate stream. The method further includes adding a second major solvent of a second polarity to the reduced first eluate stream to produce a second feedstock stream. The method includes passing the second feedstock stream through a second stationary phase to provide a second eluate stream. The second eluate stream has a higher purity of the THC than in the second feedstock stream as measured by weight percentage of the THC content. The first polarity and the second polarity are opposite.

Further and alternative aspects and features of the disclosed principles will be appreciated from the following detailed description and the accompanying drawings. As will be appreciated, the systems and techniques for purifying THC from a composition containing THC and at least one impurity that are disclosed herein are capable of being carried out and used in other and different embodiments, and capable of being modified in various respects. Accordingly, it is to be understood that both the foregoing general description and the following detailed description are exemplary and explanatory only and do not restrict the scope of the appended claims.

### BRIEF DESCRIPTION OF THE DRAWINGS

The accompanying drawings are included to provide a further understanding of the disclosure. The drawings illustrate embodiments of the disclosure and together with the description serve to explain the principles of the embodiments of the disclosure.

FIG. 1 is a schematic diagram for an OR-6 single column used for removing at least a portion of lipids/waxes, terpenes, chlorophyll and pigments, and pesticides.

FIG. 2 is a schematic diagram for an OR-3 single column used for removing at least a portion of lipids/waxes, terpenes, pigments, and pesticides.

FIG. 3 is a schematic diagram for an OR-8 single column used for removing at least a portion of chlorophyll, carbohydrates, pigments, and pesticides.

FIG. 4 is a schematic diagram for a solid liquid extraction used for removing at least a portion of lipids/waxes and terpenes.

FIG. 5 is a schematic diagram for an OR-6 single column used for removing at least a portion of lipids/waxes, terpenes, chlorophyll and pigments, and pesticides.

FIG. 6 is a schematic diagram for an OR-5 single column used for removing at least a portion of lipids/waxes, carbohydrates, terpenes, pigments, and pesticides.

FIG. 7 is a schematic diagram for an OR-10 single column used for removing at least a portion of lipids/waxes, terpenes, pigments, and pesticides.

FIG. 8 is a flow diagram showing exemplary embodiments for the purification of cannabis biomass.

FIG. 9 is a flow diagram showing exemplary embodiments for the purification of crude THC oil.

### DETAILED DESCRIPTION OF EXEMPLARY EMBODIMENTS

Cannabinoids are a family of naturally occurring C₂₁ terpenophenolic compounds uniquely produced in cannabis. Marijuana usually refers to a mixture of leaves and flowering heads of the pistillate plant of Cannabis sativa from which tetrahydrocannabinols (THCs) are isolated. THCs contain two main isomeric forms, depending on the position of the double bond. The position of the double bond and the stereochemistry of these THCs have been confirmed by nuclear magnetic resonance and X-ray structure.

Extracting active ingredients from cannabis routinely extracts a number of impurities which are difficult to remove from the finished product; and, therefore a large number of purification steps, including expensive column chromatography, are required in conventional methods to isolate components.

In various embodiments, the present disclosure relates to isolating and purifying cannabinoids from plants of the genus Cannabis, which contains three species, namely Cannabis sativa, Cannabis indica, and Cannabis ruderalis. The disclosure provides methods of extraction from the plant and purification using column chromatography. The desired extracted cannabinoid, THC, can be purified to high levels, thereby allowing for their use in various pharmaceutical and nutraceutical applications. For example, in certain aspects purified THC can be obtained, which has pesticide levels below the acceptable maximum limit.

Embodiments of a method following principles of the present disclosure can comprise extracting, purifying, and isolating THC using at least one chromatographic step (e.g., column chromatography). Any suitable adsorbent (e.g., OR-3, OR-5, OR-6, OR-8, OR-10, or a combination thereof) can be used for the chromatographic methods described herein. The adsorbent can be utilized in any suitable arrangement (e.g., single column chromatography, batch column chromatography, SMB chromatography, or a combination thereof). Embodiments of a method following principles of the present disclosure can comprise using more than one adsorbent and more than one arrangement to achieve the desired purity of THC. In embodiments, a THC product having a total tetrahydrocannabinol (THC) purity greater than 85 wt. % (e.g., greater than about 90 wt. %, greater than about 95 wt. %, greater than about 96 wt. %, greater than about 97 wt. %, greater than about 98 wt. %, greater than about 99 wt. %, or greater than about 99.9 wt. %) following evaporation or drying can be obtained. In some aspects, a THC recovery yield of greater than 50 wt. % (e.g., greater than about 60 wt. %, greater than about 70 wt. %, greater than about 80 wt. %, or greater than about 90 wt. %) following evaporation or drying can be obtained.

In various aspects, the disclosure relates to methods for purification and separation of THC from cannabis and purification of cannabinoids. Embodiments of a method following principles of the present disclosure can comprise employing chromatographic stationary phases and purification procedures for purifying and isolating THC. In various embodiments, benefits of methods following principles of the disclosure include, but are not limited to, (i) increasing yield of THC, (ii) increasing purity of THC, (iii) reducing the amount of pesticides, and/or (iv) allowing for regeneration and reuse of chromatographic stationary phases.

In some aspects of the disclosure, column chromatography (e.g., SMB, batch, or single) can be utilized with any of the unique chromatographic stationary phases (e.g., OR-3, OR-5, OR-6, OR-8, and/or OR-10) described herein to obtain an increased purity of THC. For example, the chromatographic stationary phases can be regenerated to obtain an increased yield of THC, and allow for reuse of the chromatographic stationary phases.

In additional aspects of the disclosure, batch column chromatography can be utilized to produce an increased yield of THC and increase the longevity of chromatographic stationary phases. The process reuses a chromatographic stationary phases in another stage of the purification process to obtain more THC and to increase the utility of the chromatographic stationary phases.

Embodiments of methods following principles of the present disclosure can be used to purify THC by separating a first constituent (i.e., THC) from a second constituent (e.g., at least one impurity or a second cannabinoid) so as to provide a first composition wherein the first constituent is in a higher concentration relative to a second constituent and/or a second composition wherein the second constituent is in a higher concentration relative to the first constituent. To put it another way, embodiments of a method following principles of the present disclosure can be used to separate THC and at least one impurity to produce a higher purity of THC.

The processes described herein aim to separate a first constituent (i.e., THC) and a second constituent (e.g., at least one impurity and/or a second cannabinoid) from a feedstock stream (e.g., a crude cannabis extract stream). In some embodiments, the feedstock stream (e.g., a crude cannabis extract stream) comprises THC and at least one impurity (e.g., pesticides, color bodies, acidic components, lipids, cannabis plant waxes, a second cannabinoid, or mixtures thereof) to be separated.

In some aspects, the disclosure provides a method of purifying tetrahydrocannabinol (THC) from a composition containing THC and at least one impurity, the method comprising: passing a feedstock stream comprising the composition through one or more stationary phases to provide an eluate stream having a higher purity of the THC than in the feedstock stream as measured by weight percentage of the THC content, the one or more stationary phases comprising OR-3, OR-5, OR-6, OR-8, and/or OR-10.

In some aspects, the stationary phase adsorbents may be a single stationary phase disposed in a single adsorbent bed or disposed in a single column or series of single columns (e.g., at least two columns). In other aspects, the stationary phase adsorbent may be more than one stationary phase disposed in a single adsorbent bed or disposed in a single column or series of single columns (e.g., at least two columns). Embodiments of the instant disclosure employ separate stationary phase adsorbents in carrying out the overall process of the disclosure. A list of exemplary stationary phases (i.e., chromatographic resins) for use in various embodiments of a method following principles of the present disclosure are as follows.

OR-3 is a modified hydrophilic adsorbent comprising a polar silica adsorbent having a high level of silanol (Si—O—H) groups. In some embodiments, OR-3 has an average particle diameter of from about 60 microns to about 200 microns (e.g., about 60 microns to about 150 microns, about 60 microns to about 100 microns, about 100 microns to about 200 microns, about 150 microns to about 200 microns, or about 100 microns to about 150 microns). In some embodiments, OR-3 has an average surface area of between 450 and 550 m²/g (e.g., about 450 m²/g to about 525 m²/g, about 450 m²/g to about 500 m²/g, about 475 m²/g to about 550 m²/g, or about 500 m²/g to about 550 m²/g), having an average pore volume of between 0.7 and 0.85 mL/g (e.g., about 0.7 g/mL, about 0.75 g/mL, about 0.8 g/mL, or about 0.85 g/mL). In some embodiments, OR-3 has an average pore size of between 50 to 75 Angstroms (i.e., 0.005-0.0075 microns).

OR-3 can be used to remove at least a portion of sugars, carbohydrates, pesticides, and other pigments. OR-3 can be used with polar and non-polar solvents as the mobile phase desorbent. In certain embodiments, OR-3 is used with non-polar solvents as the desorbent. The feedstock stream for OR-3 can be any suitable feedstock stream. In certain embodiments, the feedstock stream for OR-3 comprises an eluate from an OR-6 adsorbent, an eluate stream from OR-5, a crude cannabis extract, or a filtrate from a solid liquid extraction.

OR-5 is a modified hydrophobic adsorbent comprising a styrene-divinylbenzene (DVB) resin or a poly(methyl methacrylate) (PMMA) resin. In some embodiments, the styrene-divinylbenzene (DVB) resin has from about 4 to about 8% (e.g., about 4%, about 4.5%, about 5%, about 5.5%, about 6%, about 6.5%, about 7%, about 7.5%, or about 8%) crosslinking. In some embodiments, OR-5 has an average particle size range of from about 25 microns to about 300 microns (e.g., about 25 microns to about 200 microns, about 25 microns to about 100 microns, about 100 microns to about 300 microns, about 200 microns to about 300 microns, or about 50 microns to about 250 microns). In some embodiments, OR-5 has an average bulk density of from about 0.4 g/mL to about 0.6 g/mL (e.g., about 0.4 g/mL, about 0.45 g/mL, about 0.5 g/mL, about 0.55 g/mL, or about 0.6 g/mL), an average surface area of from about 450 m²/g to about 550 m²/g (e.g., about 450 m²/g to about 525 m²/g, about 450 m²/g to about 500 m²/g, about 475 m²/g to about 550 m²/g, or about 500 m²/g to about 550 m²/g). In some embodiments, OR-5 has an average pore volume of from about 0.7 mL/g to about 0.9 mL/g (e.g., about 0.7 g/mL, about 0.75 g/mL, about 0.8 g/mL, about 0.85 g/mL, or about 0.9 g/mL). In certain embodiments of OR-5 resin, the modified hydrophobic adsorbent (i.e., hydrophobic resin) is a C18 resin.

OR-5 can be used to remove at least a portion of lipids/waxes, carbohydrates, pesticides, and other pigments. OR-5 can be used with polar and non-polar solvents as the mobile phase desorbent. In certain embodiments, OR-5 is used with polar solvents as the desorbent. The feedstock stream for OR-3 can be any suitable feedstock stream. In certain embodiments, the feedstock stream for OR-5 comprises an eluate from an OR-6 adsorbent, an eluate stream from OR-5, a crude cannabis extract, or a filtrate from a solid liquid extraction. High purity THC adsorbed to single column OR-5 can be recovered using an ethanol wash. After high purity THC is desorbed from OR-5 using an ethanol wash, a complete regeneration of the stationary phase can be achieved using an acetone wash. After an acetone wash, the single column OR-5 can be reused for additional purification.

OR-6 is a modified activated carbon adsorbent which was heat treated to provide a highly hydrophobic adsorbent which is essentially free of hydroxyl groups. In some embodiments, OR-6 has an average particle size range of from about 40 to about 1700 microns (e.g., about 50 to about 1000 microns, about 50 to about 500 microns, about 100 microns to about 500 microns, about 100 microns to about 250 microns, or 177 and 250 microns). In some embodiments, OR-6 has an iodine number (a measure of the micropore content of the activated carbon) greater than about 900 mg/g (e.g., greater than about 1000 mg/g, greater than about 1250 mg/g, greater than about 1500 mg/g, or greater than about 2000 mg/g).

OR-6 can be used to remove at least a portion of lipids/waxes, chlorophyll, and pesticides. OR-6 can be used with polar and non-polar solvents as the mobile phase desorbent. In certain embodiments, OR-6 is used with non-polar solvents as the desorbent. The feedstock stream for OR-6 can be any suitable feedstock stream. In certain embodiments, the feedstock stream for OR-6 comprises an eluate from an OR-5 adsorbent, an eluate stream from OR-3, a crude cannabis extract, or a filtrate from a solid liquid extraction.

OR-8 is a stationary phase containing both OR-3 and OR-6. OR-8 can contain any suitable ratio of OR-3 and OR-6. For example, OR-8 can contain from about a 5:95 mass ratio to about a 95:5 mass ratio (e.g., about a 10:90 mass ratio to about a 90:10 mass ratio, about a 25:75 mass ratio to about a 75:25 mass ratio, about a 40:60 mass ratio to about a 60:40 mass ratio, about a 20:80 mass ratio to about a 60:40 mass ratio, or about a 40:60 mass ratio to about a 80:20 mass ratio) of OR-3 to OR-6.

OR-8 can be used to remove at least a portion of lipids/waxes, sugars, carbohydrates, chlorophyll, pesticides, and other impurities. OR-8 can be used with polar and non-polar solvents as the mobile phase desorbent. In certain embodiments, OR-8 is used with non-polar solvents as the desorbent. The feedstock stream for OR-8 can be any suitable feedstock stream. In certain embodiments, the feedstock stream for OR-8 comprises a crude cannabis extract or a filtrate from a solid liquid extraction.

OR-10 is a stationary phase containing both OR-5 and OR-6. OR-10 can contain any suitable ratio of OR-5 and OR-6. For example, OR-8 can contain from about a 5:95 mass ratio to about a 95:5 mass ratio (e.g., about a 10:90 mass ratio to about a 90:10 mass ratio, about a 25:75 mass ratio to about a 75:25 mass ratio, about a 40:60 mass ratio to about a 60:40 mass ratio, about a 20:80 mass ratio to about a 60:40 mass ratio, or about a 40:60 mass ratio to about a 80:20 mass ratio) of OR-5 to OR-6. Without wishing to be bound by any particular theory, it is believed that the OR-10 adsorbent may have the added benefit of providing a more uniform column distribution and lower pressure drop than OR-5 or OR-6 alone.

OR-10 can be used to remove at least a portion of lipids/waxes, pigments, pesticides, and other impurities. OR-10 can be used with polar and non-polar solvents as the mobile phase desorbent. In certain embodiments, OR-10 is used with polar solvents as the desorbent. The feedstock stream for OR-10 can be any suitable feedstock stream. In certain embodiments, the feedstock stream for OR-10 comprises a crude cannabis extract or a filtrate from a solid liquid extraction.

Typically, the stationary phase (i.e., adsorbent) is contained in a container (e.g., a column). The container can be any suitable container. Generally the container is a column. The chromatographic resin can be in a single column, or in more than one column (e.g., two or more columns, three or more columns, four or more columns, five or more columns, six or more columns, seven or more columns, eight or more columns, nine or more columns, or ten or more columns). In some embodiments, the stationary phase (i.e., adsorbent) is in a single column. In some embodiments, the chromatographic resin is in more than one column (e.g., two columns, three columns, or four columns, etc.).

The composition can be purified by any suitable chromatography method. For example, the composition can be purified by single column chromatography, batch column chromatography, or simulated moving bed (SMB) chromatography. In embodiments, single column chromatography comprises a purification process in which the composition is passed through a single stationary phase contained in a single container. In embodiments, batch column chromatography comprises a purification process in which the composition is passed through one or more stationary phases contained in more than one container. U.S. Pat. No. 2,985,589 describes a simulated moving bed (SMB) chromatography technique in which a chromatography system involving a separation tower is divided into a number of individual separation beds. These beds are connected in series, and the outlet at the bottom most bed is connected to a pump that returned flow in a continuous loop to the upper most bed. The inlet apparatus for each bed has a port connected to a downward flowing conduit. The conduits terminate in fittings attached to a rotary valve designed to control both ingress and egress of liquids into or from the inlets to each individual bed. The system is called Simulated Moving Bed (SMB) chromatography because the beds appear to be moving in a direction countercurrent to the direction of flow.

In some embodiments, the stationary phases described herein can be flushed with a solvent (e.g., ethanol) to recover one or more cannabinoids (e.g., THC). In some embodiments, the stationary phases described herein can be regenerated for use in subsequent separation cycles. In embodiments, regeneration can comprise washing the resin with a regeneration solution to remove the at least one impurity and/or second cannabinoid. The chromatographic resins (e.g., OR-3, OR-5, OR-6, OR-8, and/or OR-10) can be regenerated using any suitable regeneration solution. The regeneration solution of some embodiments comprises less than 5 wt. % water, and includes ethanol, acetone, or a combination thereof. In preferred embodiments, the regeneration solution comprises acetone.

The at least one impurity can be considered any compound or mixture of compounds that are not the desired target cannabinoid (i.e., THC). For example, the at least one impurity can include one or more of pesticides (e.g., bifenazate, spinomesifen, and bifenthrin), waxes, lipids, pigments, sugars, carbohydrates, proteins, chlorophyll, and mixtures thereof. In some embodiments, the at least one impurity can include other cannabinoids, e.g., a second cannabinoid, a third cannabinoid, etc., that are not the desired target cannabinoid.

In certain aspects, the at least one impurity is a pesticide (e.g., bifenazate, spinomesifen, and bifenthrin). In preferred embodiments, methods following principles of the present disclosure reduce the amount of pesticides in the composition below the maximum limit for human consumption (e.g., oral consumption by eating, smoking, or inhalation). For example, any one of bifenazate, spinomesifen, or bifenthrin can be reduced below the maximum limit for human consumption. In more preferred embodiments, all pesticides are reduced below the maximum limit for human consumption. For example, any one of bifenazate, spinomesifen, or bifenthrin can be reduced below about 2 ppm (e.g., below about 1.5 ppm, below about 1 ppm, below about 0.5 ppm, or below about 0.1 ppm). In certain embodiments, the sum total of all pesticides is reduced below about 2 ppm (e.g., below about 1.5 ppm, below about 1 ppm, below about 0.5 ppm, or below about 0.1 ppm). In certain embodiments, any one of bifenazate, spinomesifen, or bifenthrin can be reduced to undetectable amounts.

In some embodiments, the second cannabinoid is selected from Cannabidiol (CBD), Tetrahydrocannabivarin (THCV), Cannabigerol (CBG), Cannabinol (CBN), Tetrahydrocannabinolic Acid (THCA), Cannabidiolic Acid (CBDA), Cannabidivarin (CBDV), or mixtures thereof. For example, the second cannabinoid can be in the form of cannabidiolic acid (CBDA), tetrahydrocannabinolic acid (THCA), cannabigerol (CBG), cannabinol (CBN), and combinations thereof. In certain embodiments, the second cannabinoid is THCA.

The purity of a constituent (e.g., THC) can be measured by any suitable means known to a person of ordinary skill in the art. In some embodiments, the purity of a constituent (e.g., THC) is measured using high performance liquid chromatography (HPLC). In some embodiments, the purity of a constituent (e.g., THC) is measured using weight percentage of the THC content. If the weight percentage of THC content increases, the THC is considered to be more pure. If the weight percentage of THC content decreases, the THC is considered to be less pure. To illustrate, THC having a weight percentage of 15% is more pure than if it had a weight percentage of 10%. Similarly, THC having a weight percentage of 90% is more pure than if it had a weight percentage of 75%.

The term “THC content” refers to the mass of THC per volume of liquid in a given stream and is expressed as grams/Liter. The mass of the THC content in a stream is determined by subjecting a fixed volume of the sample, typically 1 ml, to an effective amount of heat, up to 80° C., at atmospheric pressure for a time sufficient to fully evaporate the sample to dryness, typically 1-2 hours.

Any suitable stationary phase adsorbent (i.e., chromatographic resin) can be used in methods of the disclosure. Methods following principles of the disclosure can use normal-phase chromatography and/or reversed-phase chromatography. In embodiments, methods following principles of the present disclosure use normal-phase chromatography which comprises a suitable chromatography method using a non-polar mobile phase. As a result, polar molecules in the non-polar mobile phase tend to adsorb to the hydrophilic stationary phase, and hydrophobic molecules in the mobile phase will pass through the column and are eluted first. In embodiments, methods following principles of the present disclosure use reversed-phase chromatography which comprises a suitable chromatography method employing a polar (e.g., aqueous) mobile phase in. As a result, hydrophobic molecules in the polar mobile phase tend to adsorb to the hydrophobic stationary phase, and hydrophilic molecules in the mobile phase will pass through the column and are eluted first. In some embodiments, methods following principles of the present disclosure use both normal-phase chromatography and reversed-phase chromatography, which are employed in tandem (e.g., in series).

In embodiments, methods following principles of the present disclosure can utilize a mobile phase desorbent (“mobile phase”) to elute the first constituent (i.e., THC) and/or second constituent (e.g., at least one impurity or a second cannabinoid) from the stationary phase. In some embodiments, the combination of the first constituent (i.e., THC), the second constituent (e.g., at least one impurity or a second cannabinoid), and the mobile phase desorbent (i.e., solvent) can be considered the feedstock stream. The mobile phase can be any suitable mobile phase capable of eluting a constituent. For example, the mobile phase can comprise water, ethanol, acetone, ethyl acetate, acetonitrile, pentanes, hexanes, heptanes, methanol, propanol, or a combination thereof. In certain embodiments, the mobile phase or solvent is selected from water, ethanol, hexanes, heptanes, and a combination thereof. For the purposes of this disclosure, water, ethanol, acetone, ethyl acetate, acetonitrile, methanol, and propanol can be considered polar solvents. For the purposes of this disclosure, pentanes, hexanes, and heptanes can be considered nonpolar solvents.

Embodiments of a method following principles of the present disclosure can use a mobile phase desorbent comprising a mixture of ethanol (e.g., food grade ethanol) and water (e.g., deionized water), or in other words, an ethanolic mixture. In embodiments, the mobile phase desorbent has a ratio of ethanol to water in a range from about 50 parts ethanol (Food grade ethanol −200 Proof) to about 50 parts water to about 90 parts ethanol to about 10 parts water (i.e., a ratio of ethanol to water in a range from about 50:50 to about 90:10). In some embodiments, the mobile phase desorbent has a ratio of ethanol to water in a range from about 50 parts ethanol to about 50 parts water to about 80 parts ethanol to about 20 parts water. In embodiments, the mobile phase desorbent has a ratio of ethanol to water of equal to or greater than 50 parts ethanol to 50 parts water. In embodiments, the mobile phase desorbent used in the method has a ratio of ethanol to water of about 50:50, about 55:45, about 60:40, about 65:35, about 70:30, about 75:25, about 80:20, about 85:15, or about 90:10.

In some embodiments, a mobile phase desorbent for use in methods described herein comprises a nonpolar solvent (e.g., pentanes, hexanes, or heptanes). The mobile phase desorbent employs any suitable percent volume/volume (% v/v) of nonpolar solvent (e.g., pentanes, hexanes, or heptanes). For example, the mobile phase desorbent can comprise from about 1% v/v to about 100% v/v, for example, from about 5% v/v to about 100% v/v, from about 10% v/v to about 100% v/v, from about 20% v/v to about 100% v/v, from about 30% v/v to about 100% v/v, from about 40% v/v to about 100% v/v, from about 50% v/v to about 100% v/v, from about 60% v/v to about 100% v/v, from about 70% v/v to about 100% v/v, from about 80% v/v to about 100% v/v, from about 90% v/v to about 100% v/v, from about 20% v/v to about 80% v/v, from about 50% v/v to about 90% v/v, or from about 10% v/v to about 50% v/v. In certain embodiments, the mobile phase desorbent employs 100% v/v nonpolar solvent (e.g., pentanes, hexanes, and/or heptanes).

In embodiments, methods of the disclosure utilize a feedstock stream (i.e., feed). The feedstock stream can be prepared by any suitable method such that it contains at least one constituent (i.e., THC) to be separated (i.e., purified). In some embodiments a procedure of feed preparation is as follows. Following harvesting and processing, the grinded cannabis is extracted with an appropriate GRAS solvent, preferably ethanol, or mixtures of ethanol and water. A number of different parameters can influence the overall yield, quality and/or purity of the desired final product. These parameters include, but are not limited to, the identity of the chosen GRAS solvent; the temperature and time at which the chosen natural solvent is used; the ratio of raw material to solvent (raw material:solvent (v/v)) that is employed; the number of successive extractions performed; the chosen method of purification of the desired products and the conditions related thereto. The skilled person will understand that these parameters are not necessarily mutually exclusive, and that a particular choice relating to one parameter may or may not affect the choice of other parameters. For example, the identity of the chosen natural solvent, and the temperature thereof, can affect the optimal ratio of raw material to solvent that is required to obtain the desired results. Following the extraction of THC from the cannabis, a crude extract stream comprising crude cannabinoids and impurities is provided in the extraction zone. The crude cannabinoid stream can be filtered to remove debris and small particles in a progressive filtration step to provide a filtered crude cannabinoid stream.

In some embodiments, the crude cannabinoids are admixed with ethanol to provide a filtered crude cannabinoid stream which comprises from about 3 wt. % to about 4 wt. % (e.g., about 3.2 wt. % to about 3.8 wt. %, about 3.4 wt. % to about 4 wt. %, about 3.2 wt. % to about 3.7 wt. % or about 3.4 wt. % to about 3.7 wt. %) total crude cannabinoids in the mixture. Preferably, the filtered crude cannabinoid stream comprises from about 3.4 wt. % to about 3.7 wt. % total cannabinoids in the mixture. The concentration of solids in the filtered crude cannabinoid stream varies from about 60 g/L to about 80 g/L (e.g., about 60 g/L, about 65 g/L, about 70 g/L, about 75 g/L, or about 80 g/L), and is preferably about 75 g/L.

In certain embodiments, the feedstock stream comprises cannabis crude extract. In embodiments, cannabis crude extract can comprise feed prepared by using ethanol solvent to extract the desired compounds from cannabis. In some embodiments, the cannabis crude extract is further mixed with water to form an ethanol/water mixture. The resulting ethanol/water mixture comprising cannabis crude extract can have an ethanol to water ratio of about 100:0, e.g., about 90:10, about 80:20, about 70:30, about 60:40, or about 50:50 or less. In preferred embodiments, the ethanol to water ratio is from about 50:50 to about 80:20.

The cannabis crude extract can be obtained by any suitable method. For example, the cannabis crude extract can be extracted from cannabis (e.g., ground, flour, trim, etc.) of the three species Cannabis sativa, Cannabis indica, and Cannabis ruderalis using a hydrocarbon solvent such as butane, a supercritical solvent such as carbon dioxide, or ethanol. In embodiments, the cannabis crude extract is obtained by extraction with butane to be carried out at above atmospheric pressure. In embodiments, the cannabis crude extract is obtained using liquid carbon dioxide (CO₂) in its super-critical range (e.g., extraction temperatures above 31° C. and pressures above 74 bar). In embodiments, the cannabis crude extract is obtained by ethanol extraction.

In certain embodiments, the feedstock stream comprises decolorized cannabis crude extract. In embodiments, decolorized cannabis crude extract can comprise feed prepared by using ethanol solvent to extract the desired compounds from cannabis. The resulting extract can be then processed through a chromatographic resin to decolorize (i.e., remove chlorophylls & pigments). In some embodiments, the decolorized cannabis crude extract is further mixed with water to form an ethanol/water mixture. The resulting ethanol/water mixture comprising decolorized cannabis crude extract can have an ethanol to water ratio of about 100:0, e.g., about 90:10, about 80:20, about 70:30, about 60:40, or about 50:50 or less. In some embodiments, the ethanol to water ratio is from about 50:50 to about 80:20.

In embodiments, the feedstock stream is decolorized and decarboxylated cannabis crude extract. In embodiments, decolorized and decarboxylated cannabis crude extract can comprise feed that is prepared by using ethanol solvent to extract the desired compounds from cannabis. The resulting extract is then processed through a chromatographic resin to decolorize by removing chlorophylls and pigments. The decolorized cannabis crude extract can be placed in a still to apply heat to activate/convert the acidic form to a decarboxylated form. In some embodiments, the decolorized and decarboxylated cannabis crude extract is further mixed with water to form an ethanol/water mixture. The resulting ethanol/water mixture comprising decolorized and decarboxylated cannabis crude extract can have an ethanol to water ratio of about 100:0, e.g., about 90:10, about 80:20, about 70:30, about 60:40, or about 50:50 or less. In preferred embodiments, the ethanol to water ratio is from about 50:50 to about 80:20.

In certain embodiments, the feedstock stream is decarboxylated cannabis crude extract. In embodiments, decarboxylated cannabis crude extract can comprise feed that is prepared by using ethanol solvent to extract the desired compounds from cannabis. The resulting extract can be placed in a still to apply heat to activate/convert the acidic form to a decarboxylated form. In some embodiments, the decarboxylated cannabis crude extract is further mixed with water to form an ethanol/water mixture. The resulting ethanol/water mixture comprising decarboxylated cannabis crude extract can have an ethanol to water ratio of about 100:0, e.g., about 90:10, about 80:20, about 70:30, about 60:40, or about 50:50 or less. In preferred embodiments, the ethanol to water ratio is from about 50:50 to about 80:20.

In embodiments, the feedstock stream comprises crude THC oil. In some embodiments, the crude THC oil is further mixed with an ethanol/water mixture. The resulting ethanol/water mixture comprising THC oil can have an ethanol to water ratio of about 100:0, e.g., about 90:10, about 80:20, about 70:30, about 60:40, or about 50:50 or less. In preferred embodiments, the ethanol to water ratio is from about 50:50 to about 80:20. In some embodiments, the crude THC oil is further mixed with a nonpolar solvent such as hexanes or heptanes.

In embodiments, the feedstock stream is decarboxylated crude THC oil. In embodiments, crude THC oil and an optional solvent can be placed in a still to apply heat to activate/convert the acidic form to a decarboxylated form. In some embodiments, the decarboxylated cannabis crude extract is further mixed with an ethanol/water mixture. The resulting ethanol/water mixture comprising decarboxylated crude THC oil can have an ethanol to water ratio of about 100:0, e.g., about 90:10, about 80:20, about 70:30, about 60:40, or about 50:50 or less. In preferred embodiments, the ethanol to water ratio is from about 50:50 to about 80:20. In some embodiments, the decarboxylated crude THC oil is further mixed with a nonpolar solvent such as hexanes or heptanes.

In some embodiments, the method further comprises one or more solid-liquid extraction steps. A solid-liquid extraction can be performed on any composition (e.g., a crude cannabis, a feedstock stream, or an eluate) or a reduced version (i.e., wherein at least some of the solvent has been removed) of any composition (e.g., a crude cannabis, a feedstock stream, or an eluate). Alternatively, or in addition to, a solid-liquid extraction can be performed to obtain any composition (e.g., a crude cannabis, a feedstock stream, or an eluate). In embodiments, solid-liquid extraction comprises precipitating a solid out of solution and removing the solid from solution (e.g., by filtering and/or decanting). In certain embodiments, a composition can be mixed with a solvent mixture (e.g., an ethanolic mixture) to remove most (e.g., all or substantially all) of the lipids and/or waxes and at least a portion of the terpenes.

In some embodiments, the method further comprises one or more liquid-liquid extraction steps. A liquid-liquid extraction can be performed on any composition (e.g., a crude cannabis, a feedstock stream, or an eluate) or a reduced version, wherein at least some of the solvent has been removed, of any composition (e.g., a crude cannabis, a feedstock stream, or an eluate). Alternatively, or in addition to, a liquid-liquid extraction can be performed to obtain any composition (e.g., a crude cannabis, a feedstock stream, or an eluate).

In some embodiments, the method further comprises one or more microfiltration steps. A microfiltration step can be performed on any composition (e.g., a crude cannabis, a feedstock stream, or an eluate) or a reduced version (i.e., wherein at least some of the solvent has been removed) of any composition (e.g., a crude cannabis, a feedstock stream, or an eluate). Alternatively, or in addition to, a solid liquid extraction can be performed to obtain any composition (e.g., a crude cannabis, a feedstock stream, or an eluate). Typically, a microfiltration step is performed after the solid liquid extraction to form a filtrate.

It will be understood to those of ordinary skill in the art that solvent can be removed (i.e., the composition can be concentrated) and/or added (i.e., the composition can be diluted) to any composition (e.g., a crude cannabis, a feedstock stream, filtrate, or an eluate) or a reduced version (i.e., wherein at least some of the solvent has been removed) of any composition (e.g., a crude cannabis, a feedstock stream, filtrate, or an eluate). The solvent can be removed or added for any suitable reason. For example, solvent can be removed or added to (i) change the polarity of the composition, (ii) obtain a dry sample of the composition, (iii) isolate the final product, or (iv) increase or decrease the loading amount for column chromatography.

In some embodiments, the method further comprises removing the solvent to provide a reduced version of the composition (e.g., a crude cannabis, a feedstock stream, filtrate, or an eluate). The solvent can be removed by any suitable method. For example, the solvent can be removed by evaporation (e.g., under reduced pressure, elevated temperature, or a combination thereof), membrane permeation (e.g., nano-filtration), or a combination thereof.

In some embodiments, the method further comprises adding solvent to provide a diluted version of the composition (e.g., a crude cannabis, a feedstock stream, filtrate, or an eluate). The solvent can be added by any suitable method.

The eluate stream from one stationary phase can be used directly as a feedstock stream for a second stationary phase. For example, in embodiments of a method following principles of the present disclosure, the feedstock stream, the stationary phase, and the eluate can comprise a first feedstock stream, a first stationary phase, and a first eluate, respectively, the method further comprising: passing the first eluate through a second stationary phase to form a second eluate having a higher purity of THC than in the first eluate as measured by weight percentage of the THC content. The method can further comprise passing the second eluate through a third stationary phase to form a third eluate having a higher purity of THC than in the second eluate as measured by weight percentage of the THC content.

The eluate stream from one stationary phase can be used as a feedstock stream for a second stationary phase after the eluate stream has undergone a solid liquid extraction. For example, in embodiments of a method following principles of the present disclosure, the feedstock stream, the stationary phase, and the eluate can comprise a first feedstock stream, a first stationary phase, and a first eluate, respectively, the method further comprising: optionally removing the solvent from the first eluate to form a reduced first eluate, performing a solid liquid extraction on the first eluate or reduced first eluate to provide a filtrate, optionally removing the solvent from the filtrate to form a reduced filtrate, and passing the filtrate or reduced filtrate through a second stationary phase to form a second eluate having a higher purity of THC than in the first eluate as measured by weight percentage of the THC content.

In an illustrative embodiment of a method following principles of the present disclosure, the feedstock stream, the stationary phase, and the eluate comprise a first feedstock stream, a first stationary phase, and a first eluate, respectively, the method further comprising: passing the first eluate through a second stationary phase to form a second eluate having a higher purity of THC than in the first eluate as measured by weight percentage of the THC content; passing a second feedstock stream through the second stationary phase to form a third eluate having a higher purity of the cannabinoid than in the second feedstock stream as measured by weight percentage of the THC content; and passing the third eluate through a third stationary phase to form a fourth eluate having a higher purity of the cannabinoid than in the third eluate as measured by weight percentage of the THC content. Without wishing to be bound by any particular theory, it is believed that an added benefit of this illustrative embodiment is that once a stationary phase is saturated with an impurity, the column may still be used to remove other impurities such as waxes, lipids, and/or pigments. In addition, any portion of THC adsorbed to the second chromatographic column may be recovered by passing a second feedstock stream through the second chromatographic resin to elute at least a portion of the adsorbed THC in the third eluate. Accordingly, such method can increase the longevity of a stationary phase, and increase the yield of the THC, under certain circumstances.

In some aspects, embodiments of a method following principles of the present disclosure can comprise purifying tetrahydrocannabinol (THC) from a composition containing THC and at least one impurity, the method comprising: passing a first feedstock stream comprising the composition through a first stationary phase to provide a first eluate stream having a higher purity of the THC than in the first feedstock stream as measured by weight percentage of the THC content, the first feedstock stream further comprising a first major solvent of a first polarity; removing at least some of the first major solvent of the first polarity from the first eluate stream to produce a reduced first eluate stream; adding a second major solvent of a second polarity to the reduced first eluate stream to produce a second feedstock stream; and passing the second feedstock stream through a second stationary phase to provide a second eluate stream having a higher purity of the THC than in the second feedstock stream as measured by weight percentage of the THC content, wherein the first polarity and the second polarity are opposite. The method can further comprise a solid liquid extraction of the first eluate stream or the reduced first eluate stream with a second major solvent to produce a filtrate, and using the filtrate as the second feedstock stream.

As used herein, the term “major solvent” refers to the chemical compound (e.g., solvent) that makes up the majority of any composition (e.g., a crude cannabis, a feedstock stream, filtrate, or an eluate).

In an illustrative embodiment, the method comprises passing a first feedstock stream comprising a composition containing THC and at least one impurity and a nonpolar solvent (e.g., pentanes, hexanes, and/or heptanes) as the first major solvent through a first stationary phase (e.g., OR-3, OR-5, OR-6, OR-8, and/or OR-10) to provide a first eluate stream having a higher purity of the THC than in the first feedstock stream as measured by weight percentage of the THC content; optionally performing a solid liquid extraction with a polar solvent (e.g., ethanol and/or water) on the first eluate stream, or a mixture formed from the first eluate stream (e.g., by reducing or adding solvent), to form a filtrate; using the first eluate stream or the filtrate as a second feedstock stream; and passing the second feedstock stream comprising containing THC and at least one impurity and a polar solvent (e.g., ethanol and/or water) as the major solvent through a second stationary phase (e.g., OR-3, OR-5, OR-6, OR-8, and/or OR-10) to provide a second eluate stream having a higher purity of the THC than in the second feedstock stream.

In another illustrative embodiment, the method comprises passing a first feedstock stream comprising a composition containing THC and at least one impurity and a polar solvent (e.g., ethanol and/or water) as the first major solvent through a first stationary phase (e.g., OR-3, OR-5, OR-6, OR-8, and/or OR-10) to provide a first eluate stream having a higher purity of the THC than in the first feedstock stream as measured by weight percentage of the THC content; optionally performing a solid liquid extraction with a nonpolar solvent (e.g., pentanes, hexanes, and/or heptanes) on the first eluate stream, or a mixture formed from the first eluate stream (e.g., by reducing or adding solvent), to form a filtrate; using the first eluate stream or the filtrate as a second feedstock stream; and passing the second feedstock stream comprising containing THC and at least one impurity and a nonpolar solvent (e.g., pentanes, hexanes, and/or heptanes) as the major solvent through a second stationary phase (e.g., OR-3, OR-5, OR-6, OR-8, and/or OR-10) to provide a second eluate stream having a higher purity of the THC than in the second feedstock stream.

In some embodiments, the methods described herein provide an isolated yield (i.e., a percent recovery) after removal of solvent of at least about 50% or more (e.g., at least about 55% or more, at least about 60% or more, at least about 65% or more, at least about 70% or more, at least about 80% or more, at least about 85% or more, at least about 90% or more, or at least about 95% or more) of THC. In preferred embodiments, the methods described herein provide an isolated yield of from about 75% to about 100% (e.g., about 75% to about 90%, about 75% to about 85%, about 80% to about 100%, about 80% to about 90%, about 85% to about 100%, or about 85% to about 90%) of THC.

In some embodiments, the methods described herein provide a THC product having a total tetrahydrocannabinol (THC) purity greater than about 85 wt. % (e.g., greater than about 86 wt. %, greater than about 87 wt. %, greater than about 88 wt. %, greater than about 89 wt. %, greater than about 90 wt. %, greater than about 95 wt. %, greater than about 96 wt. %, greater than about 97 wt. %, greater than about 98 wt. %, greater than about 99 wt. %, or greater than about 99.9 wt. %) following removal of solvent (e.g., by evaporation or drying).

Embodiments of a method following principles of the present disclosure can be performed on a laboratory scale or a commercial scale.

The disclosure is further illustrated by the following exemplary purification protocols.

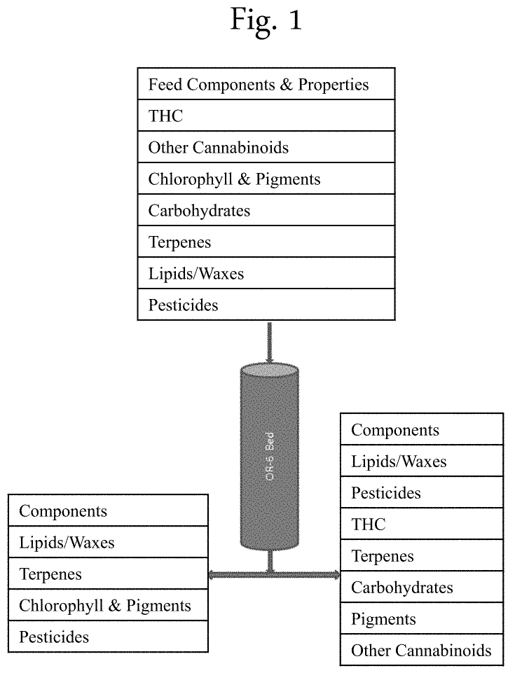

In embodiments, OR-6 can be used as a single adsorbent for decolorization. The OR-6 adsorbent can be used to purify THC by removing most (e.g., all or substantially all) of the chlorophyll and at least a portion of terpenes, pesticides, and/or lipids/waxes. As shown in FIG. 1, a cannabis crude extract, after decarboxylation (can also be carried out before decarboxylation), can be mixed with a non-polar solvent (e.g., pentanes, hexanes, or heptanes) and processed through one or more columns with OR-6 adsorbent to enrich THC with the removal of most (e.g., all or substantially all) of the chlorophyll and at least a portion of terpenes, pesticides, and/or lipids/waxes. Once the chlorophyll adsorption limit levels on the OR-6 stationary phase have been exceeded, purification using this particular adsorbent can be stopped. The non-polar solvent can be removed to provide a THC enriched oil.

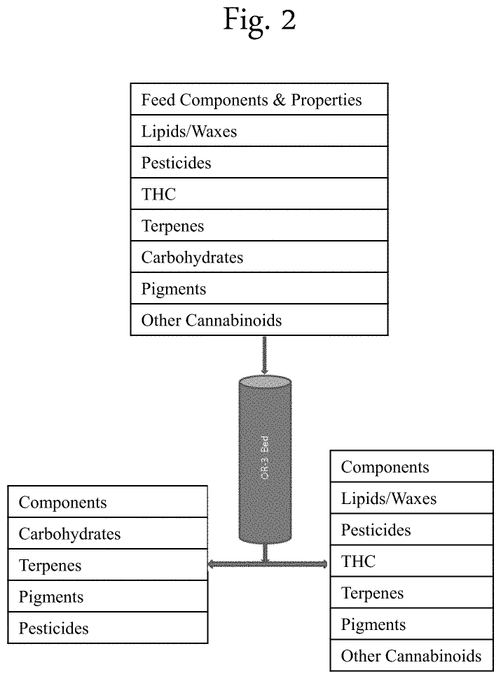

In embodiments, OR-3 can be used as a single adsorbent for carbohydrate and pigment removal. The OR-3 adsorbent can be used to purify THC by removing most (e.g., all or substantially all) of the sugars, carbohydrates, and pesticides, and at least a portion of the pigments. As shown in FIG. 2, a cannabis crude extract, after decarboxylation (can also be carried out before decarboxylation), can be mixed with a non-polar solvent (e.g., pentanes, hexanes, or heptanes) and processed through one or more columns with OR-3 adsorbent to enrich THC with the removal of most (e.g., all or substantially all) of the sugars, carbohydrates, and pesticides and at least a portion of the pigments. The non-polar solvent can be removed to provide a THC enriched oil.

In embodiments, OR-6 and OR-3 can both be used (e.g., in series) such that the eluate stream from OR-3 can be used as the feedstock stream for OR-6 or the eluate stream from OR-6 can be used as the feedstock stream for OR-3 to provide a THC enriched oil.

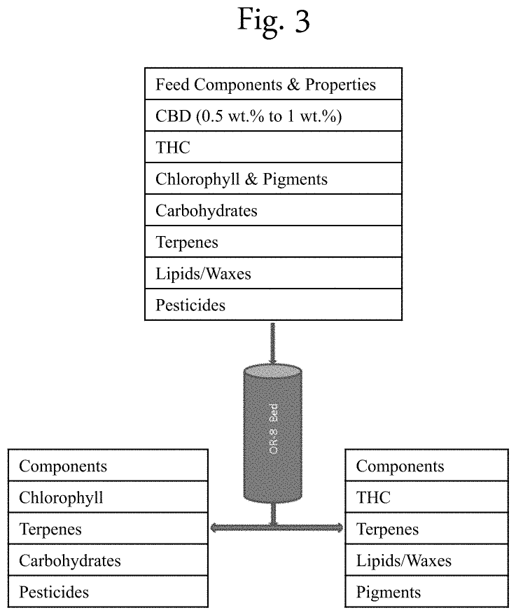

In embodiments, OR-8 can be used as a single adsorbent for decolorization and pesticide reduction. The OR-8 adsorbent can be used to purify THC by removing most (e.g., all or substantially all) of the chlorophyll, pesticides, sugars, and carbohydrates, and at least a portion of lipids/waxes and/or other pigments. As shown in FIG. 3, a cannabis crude extract, after decarboxylation (can also be carried out before decarboxylation), can be mixed with a non-polar solvent (e.g., pentanes, hexanes, or heptanes) and processed through one or more columns with OR-8 adsorbent to enrich THC with the removal of most (e.g., all or substantially all) of the chlorophyll, pesticides, sugars, and carbohydrates, and at least a portion of lipids/waxes and/or other pigments. The non-polar solvent can be removed to provide a THC enriched oil.

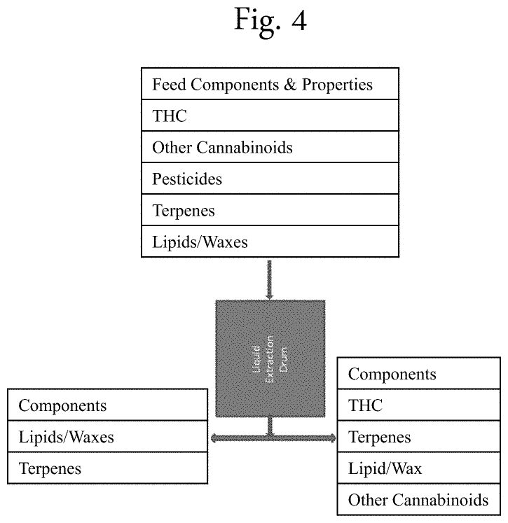

In embodiments, solid liquid extraction can be used for lipids/waxes removal (see FIG. 4). For example, the THC enriched oil from OR-3, OR-6, OR-8, or both OR-3 and OR-6 (e.g., in series) can be mixed with a solvent mixture (e.g., an ethanolic mixture) to remove most (e.g., all or substantially all) of the lipids and/or waxes and at least a portion of the terpenes. The solvent mixture can comprise from about 80% v/v to about 90% v/v ethanol and about 10% v/v to about 20% v/v water. Without wishing to be bound by any particular theory, the lipids/waxes and/or terpenes precipitate out of solution and can be removed by filtration. The solid liquid extraction produces a solid liquid extraction filtrate.

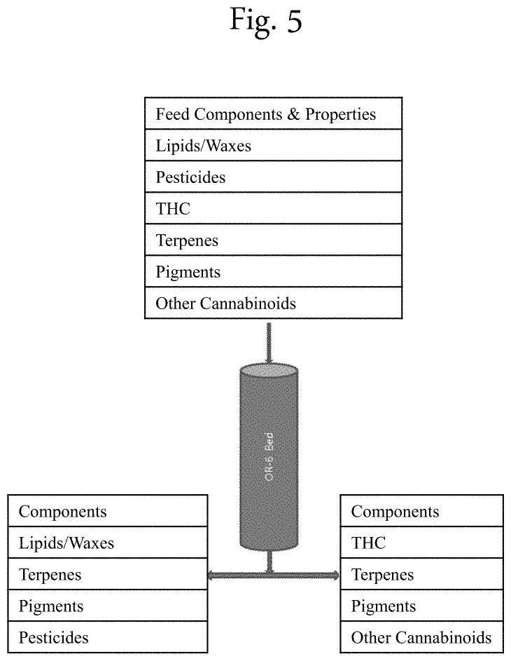

In embodiments, OR-6 can be used as a single adsorbent for polishing. The OR-6 adsorbent can be used to purify THC by removing at least a portion of lipids/waxes, pesticides, color pigments (e.g., chlorophyll), and/or other impurities from a solid liquid extraction filtrate. As shown in FIG. 5, a solid liquid extraction filtrate can be processed through one or more columns with OR-6 adsorbent to enrich THC with the removal of at least a portion of lipids/waxes, pesticides, color pigments (e.g., chlorophyll), and/or other impurities from a solid liquid extraction filtrate. The lipids/waxes, pesticides, color pigments (e.g., chlorophyll), and/or other impurities are adsorbed to the OR-6 adsorbent and the THC is passed through in the eluate. The THC purity of the resulting eluate can be as high as 85-90 wt. %.

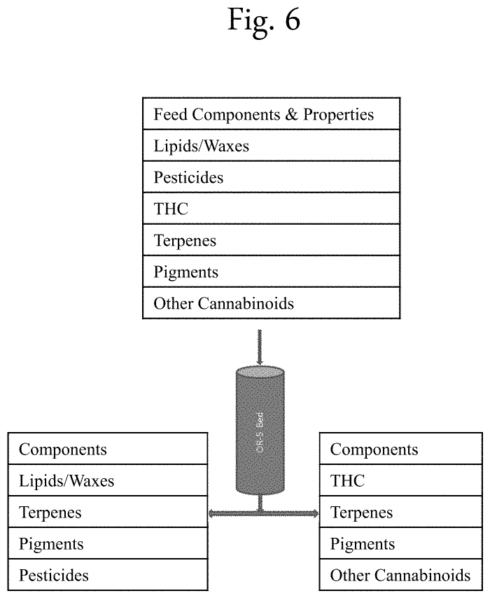

In embodiments, OR-5 can be used as a single adsorbent for polishing. The OR-5 adsorbent can be used to purify THC by removing at least a portion of lipids/waxes, pesticides, color pigments (e.g., chlorophyll), and/or other impurities from a solid liquid extraction filtrate. As shown in FIG. 6, a solid liquid extraction filtrate can be processed through one or more columns with OR-5 adsorbent to enrich THC with the removal of at least a portion of lipids/waxes, pesticides, color pigments (e.g., chlorophyll), and/or other impurities from a solid liquid extraction filtrate. THC, along with lipids/waxes, pesticides, color pigments (e.g., chlorophyll), and/or other impurities, can be adsorbed to the OR-5 adsorbent. An ethanol wash can be further performed to recover high purity THC adsorbed to the OR-5 adsorbent. The THC purity of the resulting eluate can be as high as 85-90 wt. %. An acetone wash can be performed to regenerate the OR-5 adsorbent by eluting the adsorbed lipids/waxes, pesticides, color pigments (e.g., chlorophyll), and/or other impurities.

In embodiments, OR-6 and OR-5 can both be used (e.g., in series) such that the eluate stream from OR-5 can be used as the feedstock stream for OR-6 or the eluate stream from OR-6 can be used as the feedstock stream for OR-5 to provide a THC enriched oil.

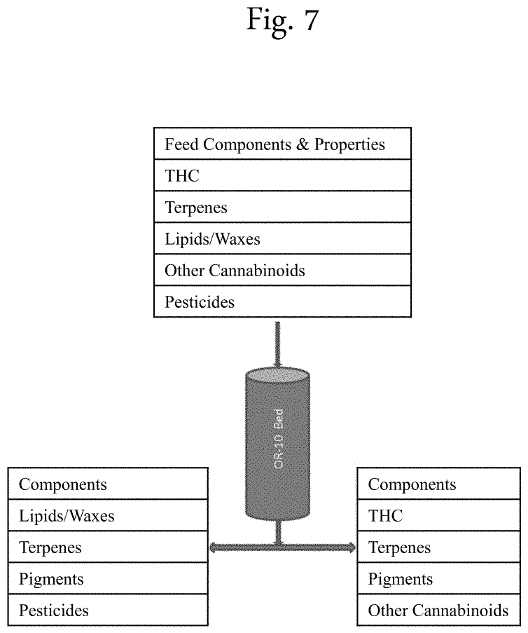

In embodiments, OR-10 can be used as a single adsorbent for polishing. The OR-10 adsorbent can be used to purify THC by removing at least a portion of lipids/waxes, pesticides, color pigments (e.g., chlorophyll), and/or other impurities from a solid liquid extraction filtrate. As shown in FIG. 7, a solid liquid extraction filtrate can be processed through one or more columns with OR-10 adsorbent to enrich THC with the removal of at least a portion of lipids/waxes, pesticides, color pigments (e.g., chlorophyll), and/or other impurities from a solid liquid extraction filtrate. The lipids/waxes, pesticides, color pigments (e.g., chlorophyll), and/or other impurities are adsorbed to the OR-10 adsorbent and the THC is passed through in the eluate. The THC purity of the resulting eluate can be as high as 85-90 wt. %.

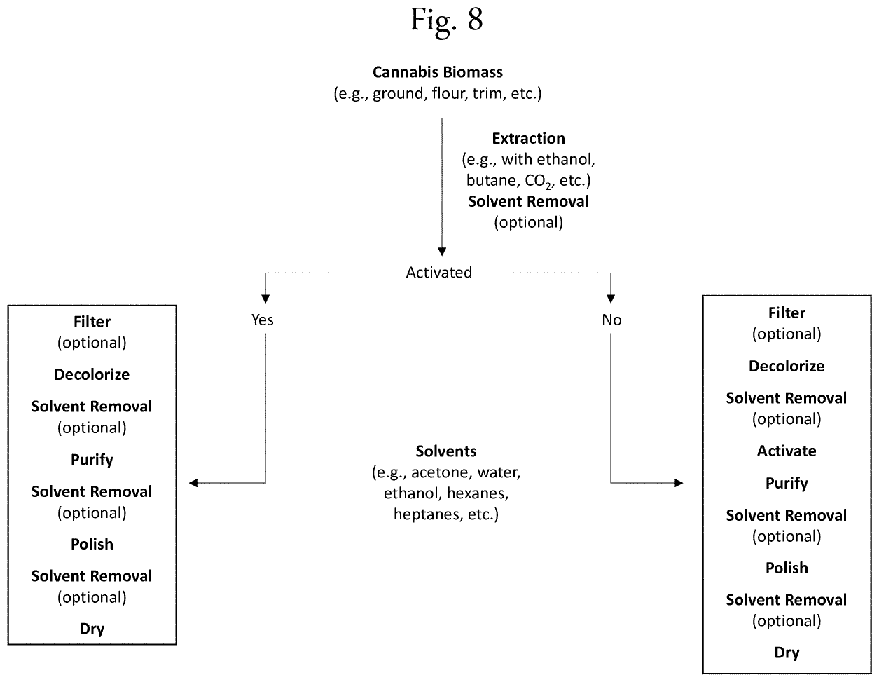

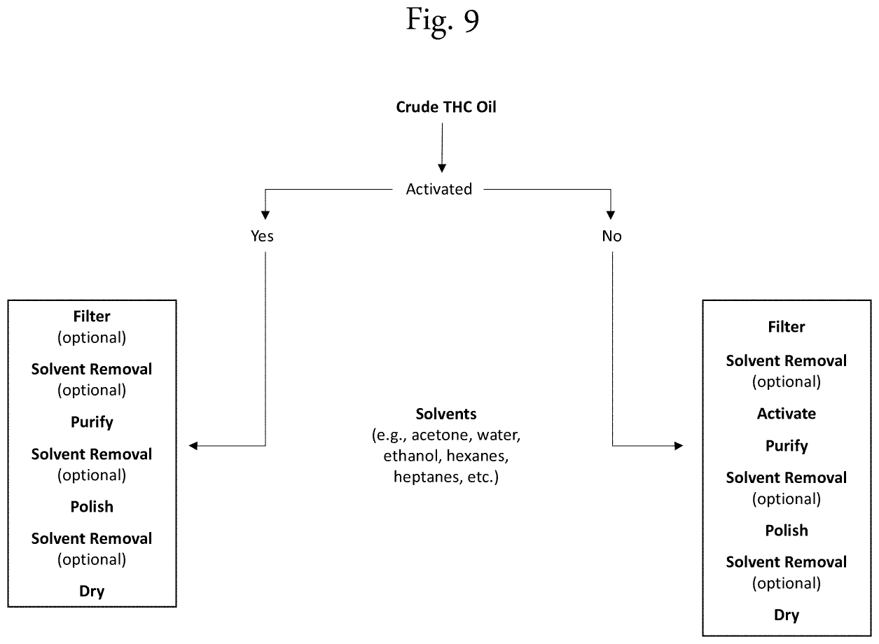

In embodiments, THC can be purified according to the flow diagram illustrated in FIG. 8. Crude cannabis extract can be obtained from extraction of cannabis biomass by any suitable method described herein. The solvent can optionally be removed from the crude cannabis extract. The resulting crude cannabis extract can optionally be activated to decarboxylate at least a portion of the acid content to form a first feedstock stream. The resulting first feedstock stream can optionally be filtered and passed through a first stationary phase to decolorize at least a portion of the first feedstock stream to form a first eluate. The solvent can optionally be removed from the resulting first eluate. If the crude cannabis extract had not been activated to decarboxylate at least a portion of the acid content to form a first feedstock stream, the first eluate can be activated at this time. The resulting first eluate can be used as a second feedstock stream and passed through a second stationary phase to form a second eluate. The solvent can optionally be removed from the second eluate. The resulting second eluate can be used as a third feedstock stream and passed through a third stationary phase to form a third eluate. The solvent can optionally be removed from the third eluate and the resulting third eluate can be dried. In embodiments, THC can be purified according to the flow diagram illustrated in FIG. 9. Crude THC oil can optionally be activated to decarboxylate at least a portion of the acid content to form an activated crude THC oil. The Crude THC oil or activated crude THC oil can optionally be filtered and the solvent can optionally be removed to form a first feedstock stream. If the crude THC oil had not been activated to decarboxylate at least a portion of the acid content the first feedstock stream can be activated at this time. The resulting first feedstock stream is passed through a first stationary phase to form a first eluate. The solvent can optionally be removed from the resulting first eluate. The resulting first eluate can be used as a second feedstock stream and passed through a second stationary phase to form a second eluate. The solvent can optionally be removed from the second eluate and the resulting second eluate can be dried.

Embodiments of a method following principles of the present disclosure can include one or more of (a)-(g) outlined below in any sequential order, or according to some embodiments the method comprises steps (a)-(g) performed sequentially in the order outlined below:
a) passing a crude cannabis extract comprising tetrahydrocannabinolic acid (THCA), tetrahydrocannabinol (THC), sugars and carbohydrates, chlorophyll and other pigments, lipids, plant waxes, impurities and other cannabinoids (e.g., 1-3% of CBDA, CBD, and/or CBN) to an activation zone and therein subjecting the crude cannabis extract to a decarboxylation reaction at a decarboxylation temperature of about 100° C. to about 120° C. (e.g., about 100° C., about 105° C., about 110° C., about 115° C., or about 120° C.) and a pressure of about −0.6 atm to about −0.75 atm (e.g., about −0.6 atm, about −0.65 atm, about −0.7 atm, or about −0.75 atm) for a decarboxylation reaction time of about 5 to about 10 hours, or sufficient time for the decarboxylation reaction to occur and proceed to completion, said decarboxylation reaction time being sufficient to fully decarboxylate essentially all of the tetrahydrocannabinolic acid (THCA) and cannabidiolic acid (CBDA) to provide a decarboxylated cannabinoid oil comprising tetrahydrocannabinol (THC), cannabidiol (CBD), lipids, plant waxes, chlorophyll and other pigments and other cannabinoids, and being essentially free of tetrahydrocannabinolic acid (THCA) and cannabidiolic acid (CBDA);
b) admixing the decarboxylated crude feed with a non-polar solvent (e.g., pentanes, hexanes, or heptanes) in the activation zone at a temperature of at least about 60° C. to form a crude cannabis extract stream, then cooling down and passing the crude cannabis extract stream comprising debris and small particles, tetrahydrocannabinol (THC), other cannabinoids, chlorophylls, color bodies, sugars and carbohydrates, lipids, plant waxes, impurities, and non-polar solvent (e.g., pentanes, hexanes, or heptanes) to a first filtration zone comprising a stainless steel strainer (cut-off size 74 μm) to remove debris and small particles to provide a filtered crude cannabinoid stream;
c) passing the filtered crude cannabinoid stream comprising tetrahydrocannabinol (THC), other cannabinols, chlorophylls, color bodies, sugars and carbohydrates, lipids, plant waxes, impurities, and non-polar solvent to a decolorization zone comprising a decolorization chromatographic column containing OR-8 adsorbent at room temperature (i.e., about 20° C.-25° C.). The combination of OR-3 adsorbent and OR-6 adsorbent remove chlorophyll, at least a portion of other color pigments, pesticides, sugars and carbohydrates, essentially all of the chlorophyll and most of the pesticides to provide a decolorized extract stream with higher purity of tetrahydrocannabinol (THC). Alternatively, the filtered crude cannabinoid stream can be passed through a chromatographic column containing OR-6 adsorbent, and then passing the OR-6 eluent through a chromatographic column containing OR-3 adsorbent in a series sequence;
d) passing the decolorized extract stream to a first evaporation zone operated at a first vacuum pressure of about −0.6 atm to about −0.75 atm (e.g., about −0.6 atm, about −0.65 atm, about −0.7 atm, or about −0.75 atm) and a temperature of about 50° C. to about 60° C. to completely remove all of the non-polar solvent to provide an evaporated extract stream which is essentially free of the non-polar solvent;
e) admixing the evaporated extract stream after decolorization with a solvent mixture having a solvent volume ratio of from about 80% v/v to about 90% v/v ethanol and about 10% v/v to about 20% v/v water at room temperature (i.e., about 20° C.-25° C.) to precipitate at least a portion of the lipids, wax and other impurities. The mixed solid and liquid is then passed through a filtration zone comprising a filtration strainer (cut-off size 74 μm), and a sequence of filtration houses with 10 microns and 1 micron pore sizes to remove the precipitate and small particles. The liquid after filtration devoid of precipitates and small particles is termed the filtrate;
f) passing the filtrate through a polishing column at a column pressure of about 2.5 atm to about 4.5 atm (e.g., about 2.5 atm, about 3 atm, about 3.5 atm, about 4 atm, or about 4.5 atm) and room temperature (i.e., about 20° C.-25° C.). The polishing chromatographic column contains OR-10 adsorbent. The combination of OR-5 adsorbent and OR-6 adsorbent remove at least a portion of the lipids, color pigment, pesticides residuals, and plant waxes to provide a stream with tetrahydrocannabinol (THC) purity greater than about 85 wt. % (e.g., greater than about 90 wt. %, greater than about 95 wt. %, greater than about 96 wt. %, greater than about 97 wt. %, greater than about 98 wt. %, greater than about 99 wt. %, greater than about 99.9 wt. %), with pesticide levels within the specification. Alternatively, one may perform the aforementioned polishing column function by passing the filtrate directly through a chromatographic column containing OR-6 adsorbent or a chromatographic column containing OR-5 adsorbent. This described polishing method does not require a “series” sequence but can be performed in series; and
g) passing the polished material through a second evaporation zone to remove mobile phase desorbent to provide a second recovered solvent stream comprising the mobile phase desorbent and to provide the high purity cannabinoid oil stream having an average tetrahydrocannabinol (THC) purity of greater than about 85 wt. % (e.g., greater than about 90 wt. %, greater than about 95 wt. %, greater than about 96 wt. %, greater than about 97 wt. %, greater than about 98 wt. %, greater than about 99 wt. %, greater than about 99.9 wt. %), with pesticide levels below the acceptable maximum limit.

In embodiments, OR-6 can be used as a single adsorbent for decolorization and lipids/waxes removal. The OR-6 adsorbent can be used to purify THC by removing most (e.g., all or substantially all) of the chlorophyll and at least a portion of terpenes, pesticides, and/or lipids/waxes (see FIG. 1). A cannabis crude extract or a solid liquid extraction filtrate, after decarboxylation (can also be carried out before decarboxylation), can be mixed with a polar solvent (e.g., alcoholic solvent) and processed through one or more columns with OR-6 adsorbent to enrich THC with the removal of most (e.g., all or substantially all) of the chlorophyll and at least a portion of terpenes, pesticides, and/or lipids/waxes. Once the chlorophyll adsorption limit levels on the OR-6 adsorbent have been exceeded, purification using this particular adsorbent can be stopped. A non-polar solvent was can be further performed to recover additional THC adsorbed to the OR-6 adsorbent.

In a further embodiment, OR-5 can be used as a single adsorbent for polishing the eluate from the OR-6 adsorbent. The OR-5 adsorbent can be used to purify THC by removing at least a portion of lipids/waxes, pesticides, color pigments (e.g., chlorophyll), and/or other impurities. As shown in FIG. 6, the eluate from the OR-6 adsorbent can be processed through one or more columns with OR-5 adsorbent to enrich THC with the removal of at least a portion of lipids/waxes, pesticides, color pigments (e.g., chlorophyll), and/or other impurities from a solid liquid extraction filtrate. The lipids/waxes, pesticides, color pigments (e.g., chlorophyll), and/or other impurities are adsorbed to the OR-5 adsorbent and the THC is passed through in the eluate. The THC purity of the resulting eluate can be as high as 85-90 wt. %. An ethanol wash can be further performed to recover additional THC adsorbed to the OR-5 adsorbent. An acetone wash can be performed to regenerate the OR-5 adsorbent by eluting the adsorbed lipids/waxes, pesticides, color pigments (e.g., chlorophyll), and/or other impurities.

OR-3 can be used as a single adsorbent for carbohydrate and pigment removal of the eluate from the OR-5 adsorbent once the solvent is removed. The OR-3 adsorbent can be used to purify THC by removing most (e.g., all or substantially all) of the sugars, carbohydrates, and pesticides, and at least a portion of the pigments. As shown in FIG. 2, the solvent reduced eluate from the OR-5 adsorbent can be mixed with a non-polar solvent (e.g., pentanes, hexanes, or heptanes) and processed through one or more columns with OR-3 adsorbent to enrich THC with the removal of most (e.g., all or substantially all) of the sugars, carbohydrates, and pesticides and at least a portion of the pigments. The non-polar solvent can be removed to provide a THC enriched oil.

According to other embodiments following principles of the disclosure, the method can include one or more of (h)-(o) outlined below in any sequential order. According to some embodiments, the method comprises steps (h)-(o) performed sequentially in the order outlined below.
h) passing a crude cannabis extract comprising tetrahydrocannabinolic acid (THCA), tetrahydrocannabinol (THC), sugars and carbohydrates, chlorophyll and other pigments, lipids, plant waxes, impurities and other cannabinoids (e.g., 1-3% of CBDA, CBD, and/or CBN) to an activation zone and therein subjecting the crude cannabis extract to a decarboxylation reaction at a decarboxylation temperature of about 100° C. to about 120° C. (e.g., about 100° C., about 105° C., about 110° C., about 115° C., or about 120° C.) and a pressure of about −0.6 atm to about −0.75 atm (e.g., about −0.6 atm, about −0.65 atm, about −0.7 atm, or about −0.75 atm) for a decarboxylation reaction time of about 5 to about 10 hours, or sufficient time for the decarboxylation reaction to occur and proceed to completion, said decarboxylation reaction time being sufficient to fully decarboxylate essentially all of the tetrahydrocannabinolic acid (THCA) and cannabidiolic acid (CBDA) to provide a decarboxylated cannabinoid oil comprising tetrahydrocannabinol (THC), cannabidiol (CBD), lipids, plant waxes, chlorophyll and other pigments and other cannabinoids, and being essentially free of tetrahydrocannabinolic acid (THCA) and cannabidiolic acid (CBDA);
i) admixing the decarboxylated crude cannabis extract with an alcohol (e.g., ethanol) or alcohol-water (e.g., ethanolic) mixture and passing the decarboxylated crude cannabis extract stream comprising debris and small particles, tetrahydrocannabinol (THC), other cannabinoids, chlorophylls, color bodies, sugars and carbohydrates, lipids, and plant waxes through a filtration zone to remove the precipitates and small particles, producing a filtered crude cannabinoid stream;
j) passing the filtered crude cannabinoid stream comprising tetrahydrocannabinol (THC), other cannabinoids, chlorophylls, color bodies, sugars and carbohydrates, lipids, and plant waxes through a decolorization zone comprising a chromatographic column containing OR-6 adsorbent at room temperature (e.g., about 20° C.-25° C.) to form a decolorized stream;
k) passing the decolorized stream (e.g., OR-6 eluate) through a chromatographic column at a column pressure of about 2.5 atm to about 4.5 atm (e.g., about 2.5 atm, about 3 atm, about 3.5 atm, about 4 atm, or about 4.5 atm) and room temperature (i.e., about 20° C.-25° C.). The chromatographic column contains OR-5 adsorbent. The OR-5 adsorbent removes the lipids/waxes, most of the color pigment (either hydrophilic or hydrophobic), sugars, carbohydrates, and at least one portion of pesticides residuals to provide a stream with tetrahydrocannabinol (THC) purity between 80 wt. % and 85 wt. %;
l) passing the OR-5 eluent stream through a first evaporation zone operated at a first vacuum pressure of about −0.6 atm to about −0.75 atm (e.g., about −0.6 atm, about −0.65 atm, about −0.7 atm, or about −0.75 atm) and a temperature of about 50° C. to about 60° C. to completely remove all of the solvent to provide an evaporated extract oil stream;
m) admixing the evaporated extract oil stream with a non-polar solvent (e.g., pentanes, hexanes, or heptanes) and passing resulting mixture through a filtration strainer (cut-off size 74 μm), and a sequence of filtration houses with 10 microns and 1-micron pore sizes to remove the precipitate and small particles. The final non-polar material free of debris and small particles is termed the filtrate;
n) passing the filtrate through a chromatographic column containing OR-3 adsorbent to remove the sugars, carbohydrates, additional pigments, and a substantial portion of the remaining pesticides, providing an OR-3 eluent stream;
o) passing the OR-3 eluent stream through a second evaporation zone to remove the mobile phase desorbent to provide a second recovered solvent stream comprising the mobile phase desorbent (alcohol or alcohol-water mixture), and to provide the high purity cannabinoid oil stream having an average tetrahydrocannabinol (THC) purity of greater than about 85 wt. % (e.g., greater than about 90 wt. %, greater than about 95 wt. %, greater than about 96 wt. %, greater than about 97 wt. %, greater than about 98 wt. %, greater than about 99 wt. %, greater than about 99.9 wt. %), with pesticide levels below the acceptable maximum limit.

In another embodiment, the disclosure includes the steps of extracting THC from crude cannabis. The steps of the leaf extraction comprise:
i) combining cannabis buds and/or cannabis leaves with a first portion of food grade ethanol to provide a first leaf/solvent mixture and agitating the first leaf/solvent mixture;
ii) soaking the first leaf/solvent mixture for an effective soaking time to form a first ethanol layer;
iii) decanting the first ethanol layer to provide a first decant stream and a first portion of wet leaves;
iv) combining a second portion of food grade ethanol with the first portion of wet leaves to provide a second leaf/solvent mixture and agitating the second leaf/solvent mixture and decanting a second ethanol layer to provide a second decant stream and residual leaves; and,
v) pressing the residual leaves to provide a third decant stream and combining the first decant stream, the second decant stream and the third decant streams to provide the crude cannabis extract stream.

The leaf extraction process is carried out at atmospheric pressure and room temperature of about 25° C. The first leaf mixture is allowed to soak for an effective soaking time comprising about 8 to 12 hours. Preferably, the combined decant streams should have a solids concentration of between about 23 to about 30 g/Liter. More preferably the combined decant streams should have a maximum solids concentration less than about 30 g/Liter.

### EMBODIMENTS

Principles of the present disclosure are incorporated in the following embodiments:

### Embodiment 1

A method of purifying tetrahydrocannabinol (THC) from a composition containing THC and at least one impurity, the method comprising:

passing a feedstock stream comprising the composition through one or more stationary phases to provide an eluate stream having a higher purity of the THC than in the feedstock stream as measured by weight percentage of the THC content,

the one or more stationary phases comprising:

a first adsorbent comprising a silica adsorbent having Si—OH groups and an average particle diameter between 60-200 microns;

a second adsorbent comprising a modified hydrophobic adsorbent having an average bulk density of from about 0.4 g/mL to about 0.6 g/mL, the modified hydrophobic adsorbent comprising at least one of a styrene-divinylbenzene (DVB) resin or a poly(methyl methacrylate) (PMMA) resin;

a third adsorbent comprising a modified activated carbon adsorbent having an average particle size range of from about 40 to about 1700 microns;

or a mixture thereof.

### Embodiment 2

The method of Embodiment 1, wherein said at least one impurity comprises at least one of pesticides, waxes, lipids, pigments, sugars, carbohydrates, proteins, chlorophyll, and mixtures thereof.

### Embodiment 3

The method of Embodiment 1 or Embodiment 2, wherein said at least one impurity comprises a second cannabinoid selected from cannabidiol (CBD), cannabidiolic Acid (CBDA), cannabigerol (CBG), cannabinol (CBN), tetrahydrocannabinolic acid (THCA), and combinations thereof.

### Embodiment 4

The method of Embodiment 1, wherein the one or more stationary phases comprises the first adsorbent.

### Embodiment 5

The method of Embodiment 1, wherein the one or more stationary phases comprises the second adsorbent.

### Embodiment 6

The method of Embodiment 1, wherein the one or more stationary phases comprises the third adsorbent.

### Embodiment 7

The method of Embodiment 1, wherein the one or more stationary phases comprises a mixture of the first adsorbent and the third adsorbent.

### Embodiment 8

The method of Embodiment 1, wherein the one or more stationary phases comprises a mixture of the second adsorbent and the third adsorbent.

### Embodiment 9

The method of Embodiment 4, wherein the first adsorbent further comprises an average surface area of from about 450 m²/g to about 550 m²/g, and an average pore volume of from about 0.7 mL/g to about 0.9 mL/g.

### Embodiment 10

The method of Embodiment 5, wherein the second adsorbent further comprises an average particle diameter of from about 25 microns to about 300 microns, an average surface area of from about 450 m²/g to about 550 m²/g, and an average pore volume of from about 0.7 mL/g to about 0.9 mL/g.

### Embodiment 11

The method of Embodiment 6, wherein the third adsorbent further comprises an iodine number greater than about 900.

### Embodiment 12

The method of any one of Embodiments 1-11, further comprising:

passing a feedstock stream comprising the composition through one stationary phase disposed in a single column to provide an eluate stream having a higher purity of the THC than in the feedstock stream as measured by weight percentage of the THC content.

### Embodiment 13

The method of any one of Embodiments 1-11, further comprising:

passing a feedstock stream comprising the composition through one stationary phase disposed in at least two columns to provide an eluate stream having a higher purity of the THC than in the feedstock stream as measured by weight percentage of the THC content.

### Embodiment 14

The method of any one of Embodiments 1-11, further comprising:

passing a feedstock stream comprising the composition through more than one stationary phase disposed in at least two columns to provide an eluate stream having a higher purity of the THC than in the feedstock stream as measured by weight percentage of the THC content.

### Embodiment 15

The method of any one of Embodiments 1-14, further comprising:

obtaining the composition from a crude cannabis extract.

### Embodiment 16

The method of Embodiment 15, wherein the crude cannabis extract is decarboxylated.

### Embodiment 17

The method of any one of Embodiments 1-14, further comprising:

obtaining the composition from a crude THC oil.

### Embodiment 18

The method of Embodiment 17, wherein the crude THC oil is decarboxylated.

### Embodiment 19

The method of any one of Embodiments 1-14, further comprising:

obtaining the composition from a step comprising performing a solid liquid extraction on a composition precursor.

### Embodiment 20

The method of any one of Embodiments 1-14, further comprising:

obtaining the composition from a step comprising microfiltering a composition precursor.

### Embodiment 21

The method of any one of Embodiments 1-14, further comprising:

obtaining the composition from a step comprising performing column chromatography on a composition precursor.

### Embodiment 22

The method of any one of Embodiments 15-21, further comprising:

obtaining the composition from a step comprising diluting or concentrating a composition precursor.

### Embodiment 23

The method of Embodiment 13, wherein at least two columns of said at least two columns are arranged in a SMB configuration to form a SMB zone, and wherein passing a feedstock stream comprising the composition through one stationary phase comprises passing the feedstock stream through the SMB zone.

### Embodiment 24

The method of Embodiment 12, wherein the feedstock stream, the stationary phase, and the eluate comprise a first feedstock stream, a first stationary phase, and a first eluate, respectively, the method further comprising:

passing the first eluate through a second stationary phase to form a second eluate having a higher purity of THC than in the first eluate as measured by weight percentage of the THC content.

### Embodiment 25

The method of Embodiment 24, further comprising:

passing the second eluate through a third stationary phase to form a third eluate having a higher purity of THC than in the second eluate as measured by weight percentage of the THC content.

### Embodiment 26

The method of Embodiment 12, wherein the feedstock stream, the stationary phase, and the eluate comprise a first feedstock stream, a first stationary phase, and a first eluate, respectively, the method further comprising:

passing the first eluate through a second stationary phase to form a second eluate having a higher purity of THC than in the first eluate as measured by weight percentage of the THC content;

passing a second feedstock stream through the second stationary phase to form a third eluate having a higher purity of the cannabinoid than in the second feedstock stream as measured by weight percentage of the THC content; and passing the third eluate through a third stationary phase to form a fourth eluate having a higher purity of the cannabinoid than in the third eluate as measured by weight percentage of the THC content.

### Embodiment 27

The method of any one of Embodiments 1-26, wherein the feedstock stream further comprises a solvent selected from water, ethanol, acetone, ethyl acetate, acetonitrile, pentanes, hexanes, heptanes, methanol, propanol, and a combination thereof.

### Embodiment 28

The method of Embodiment 27, wherein the solvent is selected from water, ethanol, hexanes, heptanes, and a combination thereof.

### Embodiment 29

The method of Embodiment 1, wherein the feedstock stream and the eluate stream comprise a first feedstock stream and a first eluate, respectively, the method further comprising:

regenerating the one or more stationary phases for passing a second feedstock stream through the regenerated one or more stationary phases to provide a second eluate stream, the second eluate stream having a higher purity of the cannabinoid than in the second feedstock stream as measured by weight percentage of the THC content.

### Embodiment 30

The method of Embodiment 29, wherein regenerating the one or more stationary phases includes washing the one or more stationary phases with a regeneration solution comprising ethanol, acetone, or a combination thereof.

### Embodiment 31

The method of Embodiment 30, wherein the regeneration solution comprises less than 5 wt. % water.

### Embodiment 32

The method of any one of Embodiments 1-31, wherein the composition comprises a first amount of pesticides and the eluate stream, the first eluate stream, the second eluate stream, or the third eluate stream has a second amount of pesticides, the second amount of pesticides below a predetermined limit for human consumption.

### Embodiment 33

A method of purifying tetrahydrocannabinol (THC) from a composition containing THC and at least one impurity, the method comprising:

passing a first feedstock stream comprising the composition through a first stationary phase to provide a first eluate stream having a higher purity of the THC than in the first feedstock stream as measured by weight percentage of the THC content, the first feedstock stream further comprising a first major solvent of a first polarity;

removing at least some of the first major solvent of the first polarity from the first eluate stream to produce a reduced first eluate stream;

adding a second major solvent of a second polarity to the reduced first eluate stream to produce a second feedstock stream; and

passing the second feedstock stream through a second stationary phase to provide a second eluate stream having a higher purity of the THC than in the second feedstock stream as measured by weight percentage of the THC content;

wherein the first polarity and the second polarity are opposite.

### Embodiment 34

The method of Embodiment 33, wherein said at least one impurity comprises at least one of pesticides, waxes, lipids, pigments, sugars, carbohydrates, proteins, chlorophyll, and mixtures thereof.

### Embodiment 35

The method of Embodiment 33 or Embodiment 34, wherein said at least one impurity comprises a second cannabinoid selected from cannabidiol (CBD), cannabidiolic Acid (CBDA), cannabigerol (CBG), cannabinol (CBN), tetrahydrocannabinolic acid (THCA), and combinations thereof.

### Embodiment 36

wherein the first stationary phase and the second stationary phase are each independently selected from:

a first adsorbent comprising a silica adsorbent having Si—OH groups and an average particle diameter between 60-200 microns;

a second adsorbent comprising a modified hydrophobic adsorbent having an average bulk density of from about 0.4 g/mL to about 0.6 g/mL, the modified hydrophobic adsorbent comprising at least one of a styrene-divinylbenzene (DVB) resin or a poly(methyl methacrylate) (PMMA) resin;

a third adsorbent comprising a modified activated carbon adsorbent having an average particle size range of from about 40 to about 1700 microns;

or a mixture thereof.

### Embodiment 37

The method of Embodiment 36, wherein the first stationary phase and/or the second stationary phase comprises the first adsorbent.

### Embodiment 38

The method of Embodiment 36, wherein the first stationary phase and/or the second stationary phase comprises the second adsorbent.

### Embodiment 39

The method of Embodiment 36, wherein the first stationary phase and/or the second stationary phase comprises the third adsorbent.

### Embodiment 40

The method of Embodiment 36, wherein the first stationary phase and/or the second stationary phase comprises a mixture of the first adsorbent and the third adsorbent.

### Embodiment 41

The method of Embodiment 36, wherein the first stationary phase and/or the second stationary phase comprises a mixture of the second adsorbent and the third adsorbent.

### Embodiment 42

The method of Embodiment 37, wherein the first adsorbent further comprises an average surface area of from about 450 m²/g to about 550 m²/g, and an average pore volume of from about 0.7 mL/g to about 0.9 mL/g.

### Embodiment 43

The method of Embodiment 38, wherein the second adsorbent further comprises an average particle diameter of from about 25 microns to about 300 microns, an average surface area of from about 450 m²/g to about 550 m²/g, and an average pore volume of from about 0.7 mL/g to about 0.9 mL/g.

### Embodiment 44

The method of Embodiment 39, wherein the third adsorbent further comprises an iodine number greater than about 900.

### Embodiment 45

The method of any one of Embodiments 33-44, wherein the first stationary phase and the second stationary phase are each respectively disposed in a single column.

### Embodiment 46

The method of any one of Embodiments 33-44, wherein at least one of the first stationary phase and the second stationary phase is respectively disposed in at least two columns.

### Embodiment 47

The method of any one of Embodiments 33-46, wherein the first major solvent and the second major solvent are each independently selected from water, ethanol, acetone, ethyl acetate, acetonitrile, pentanes, hexanes, heptanes, methanol, and propanol.

### Embodiment 48

The method of Embodiment 47, wherein the first major solvent is selected from ethanol or water and the second major solvent is selected from hexanes or heptanes.

### Embodiment 49

The method of Embodiment 47, wherein the first major solvent is selected from hexanes or heptanes and the second major solvent is selected from ethanol or water.

### Embodiment 50

The method of any one of Embodiments 33-49, further comprising:

obtaining the composition from a crude cannabis extract.

### Embodiment 51

The method of Embodiment 50, wherein the crude cannabis extract is decarboxylated.

### Embodiment 52

The method of any one of Embodiments 33-49, further comprising:

obtaining the composition from a crude THC oil.

### Embodiment 53

The method of Embodiment 52, wherein the crude THC oil is decarboxylated.

### Embodiment 54

The method of any one of Embodiments 33-49, further comprising:

obtaining the composition from a step comprising performing a solid liquid extraction on a composition precursor.

### Embodiment 55

The method of any one of Embodiments 33-49, further comprising:

obtaining the composition from a step comprising microfiltering a composition precursor.

### Embodiment 56

The method of any one of Embodiments 33-49, further comprising:

obtaining the composition from a step comprising performing column chromatography on a composition precursor.

### Embodiment 57

The method of any one of Embodiments 54-56, further comprising:

obtaining the composition from a step comprising diluting or concentrating a composition precursor.

### Embodiment 58

The method of any one of Embodiments 33-57, further comprising:

a step comprising performing a solid liquid extraction on the first eluate stream or the reduced first eluate stream.

### Embodiment 59

The method of any one of Embodiments 33-57, further comprising:

a step comprising microfiltering the first eluate stream or the reduced first eluate stream.

### Embodiment 60

The method of any one of Embodiments 33-57, further comprising:

a step comprising performing column chromatography on the first eluate stream or the reduced first eluate stream.

### Embodiment 61

The method of any one of Embodiments 33-60, wherein the composition comprises a first amount of pesticides and the second eluate stream has a second amount of pesticides, the second amount of pesticides below a predetermined limit for human consumption.

The foregoing exemplary embodiments of the disclosure numbered 1-61 are non-limiting. Other exemplary embodiments are apparent from the entirety of the description herein. As will be apparent to those of skill in the art upon reading this disclosure, each of the individually numbered embodiments may be used or combined with any of the preceding or following individually numbered aspects.

### EXAMPLES

The following examples are provided to illustrate the present disclosure. These examples are shown for illustrative purposes, and any disclosures embodied therein should not be limited thereto.

### Example 1—Decolorization Using OR-6 Adsorbent and Non-Polar Solvent

Crude cannabis extract was decarboxylated at a temperature of 100° C. to 120° C. and a pressure of −0.6 atm to −0.74 atm for a period of 5 to 10 hours to produce 97.96 g of decarboxylated crude cannabis extract. The decarboxylated crude cannabis extract was solubilized in heptanes (91.3 mg/mL) and purified using a lab scale preparation chromatography setup. The lab scale preparation chromatography setup contained two chromatographic columns, each 22 mm in diameter and 300 mm in length, connected in series and equipped with OR-6 adsorbent. The two chromatographic columns were connected to a single pump with a flow rate of 5 mL/min and heptanes as the mobile phase. The purification process was maintained at a temperature of 25° C., and the solvent was removed from the resulting eluate. The results of the purification process are set forth in Table 1.

TABLE 1
Effect on Decolorization with OR-6
Adsorbent and Non-Polar Solvent

| | Total Mass | THC Mass | THC Purity |
|---|---|---|---|
| Stream | (g) | (g) | (wt. %) |
| Decarboxylated crude | 97.96 | 42.84 | 43.7 |
| cannabis extract | | | |
| OR-6 eluate | 80.54 | 39.38 | 48.9 |

As is apparent from the results set forth in Table 1, purification with OR-6 adsorbent reduced the overall mass of the decarboxylated crude cannabis extract by 17.42 grams, while maintaining the majority of the THC mass. As a result, the overall purity of the THC increased from 43.7 wt. % to 48.9 wt. %.

### Example 2—Pigments and Carbohydrate Removal Using OR-3 Adsorbent

The OR-6 eluate from Example 1 was solubilized in heptanes (20.2 mg/mL) and purified using a lab scale preparation chromatography setup. The lab scale preparation chromatography setup contained two chromatographic columns, each 22 mm in diameter and 300 mm in length, connected in series and equipped with OR-3 adsorbent. The two chromatographic columns were connected to a single pump with a flow rate of 10 mL/min and heptanes as the mobile phase. The purification process was maintained at a temperature of 25° C., and the solvent was removed from the resulting eluate. The results of the purification process are set forth in Table 2. In addition, the amount of pesticides present in the OR-3 eluate were measured and compared to the amount of pesticides present in the crude cannabis extract used for Example 1. The results are set forth in Table 3.

TABLE 2
Effect on Pigments and Carbohydrate
Removal with OR-3 Adsorbent

| | Total Mass | THC Mass | THC Purity |
|---|---|---|---|
| Stream | (g) | (g) | (wt. %) |
| OR-6 eluate | 80.54 | 39.38 | 48.9 |
| OR-3 eluate | 47.24 | 31.8 | 67.3 |

TABLE 3
Effect on Pesticide Removal with OR-3 Adsorbent

| | Bifenazate | Spiromesifen | Bifenthrin |
|---|---|---|---|
| Stream | (ppm) | (ppm) | (ppm) |
| Crude cannabis extract | 4.6 | 8.7 | 62.0 |
| OR-3 eluate | undetectable | 0.024 | 1.5 |

As is apparent from the results set forth in Table 2, purification with OR-3 adsorbent reduces the overall mass of the OR-6 eluate by 33.3 grams, while maintaining the majority of the THC mass. As a result, the overall purity of the THC increased from 48.9 wt. % to 67.3 wt. %. In addition, Table 3 shows that purification with OR-6 adsorbent followed by OR-3 adsorbent reduces certain pesticide content to undetectable amounts.

### Example 3—Decolorization Using OR-8 Adsorbent

Crude cannabis extract was decarboxylated at a temperature of 100° C. to 120° C. and a pressure of −0.6 atm to −0.74 atm for a period of 5 to 10 hours to produce 54.5 g of decarboxylated crude cannabis extract. The decarboxylated crude cannabis extract was solubilized in heptanes (250 mg/mL) and purified using a commercial scale preparation chromatography setup. The commercial scale preparation chromatography setup contained one chromatographic column, 18 inches in diameter and 60 inches in length, equipped with OR-8 adsorbent. The chromatographic column was connected to a single pump with a flow rate of 1.5-2 L/min and heptanes as the mobile phase. The purification process was maintained at a temperature of 25° C., and the solvent was removed from the resulting eluate. The results of the purification process are set forth in Table 4.

TABLE 4
Effect on Decolorization with OR-8 Adsorbent

| | Total Mass | THC Mass | THC Purity |
|---|---|---|---|
| Stream | (g) | (g) | (wt. %) |
| Decarboxylated crude | 54.5 | 26.16 | 48 |
| cannabis extract | | | |
| OR-8 eluate | 35.67 | 23.90 | 67 |

As is apparent from the results set forth in Table 4, purification with OR-8 adsorbent reduced the overall mass of the decarboxylated crude cannabis extract by 18.83 grams, while maintaining the majority of the THC mass. As a result, the overall purity of the THC increased from 48 wt. % to 67 wt. %.

### Example 4—Lipids/Waxes Removal Using Solid Liquid Extraction

The concentrated OR-3 eluate (47.24 g) produced in Example 2 was mixed with 2 liters of an 85:15 ratio of ethanol to water mixture. The resulting solid liquid mixture was filtered using 10 μm filter paper and a Buchner funnel, and the solvent was removed from the resulting filtrate. The resulting precipitate and filtrate were analyzed and the results are set forth in Table 5.

TABLE 5
Effect on Lipids/Waxes Removal Using Solid Liquid Extraction

| | Total Mass | THC Mass | THC Purity |
|---|---|---|---|
| Stream | (g) | (g) | (wt. %) |
| OR-3 eluate | 47.24 | 31.80 | 67.3 |
| Solid liquid extraction | 40.81 | 31.09 | 76.2 |
| filtrate | | | |
| Solid liquid extraction | 6.43 | 0.72 | 6.4 |
| precipitate | | | |

As is apparent from the results set forth in Table 5, solid liquid extraction of the OR-3 eluate reduced the overall mass by 6.43 grams, while maintaining the majority of the THC mass. Thus, very little THC mass is lost to the solid liquid extraction precipitate. As a result, the overall purity of the THC increased from 67.3 wt. % to 76.2 wt. %.

### Example 5—Polishing Using OR-6 Adsorbent

The filtrate from the solid liquid extraction of Example 4 was solubilized in an ethanolic mixture (20.4 mg/mL) and purified using a lab scale preparation chromatography setup. The lab scale preparation chromatography setup contained one chromatographic column, 22 mm in diameter and 300 mm in length, equipped with OR-6 adsorbent. The chromatographic column was connected to a single pump with a flow rate of 5.0 mL/min and an ethanolic mixture as the mobile phase. The purification process was maintained at a temperature of 25° C., and the solvent was removed from the resulting eluate. The results of the purification process are set forth in Table 6.

TABLE 6
Effect on Polishing with OR-6 Adsorbent

| | Total Mass | THC Mass | THC Purity |
|---|---|---|---|
| Stream | (g) | (g) | (wt. %) |
| Solid liquid extraction | 40.81 | 31.09 | 76.2 |
| filtrate | | | |
| OR-6 eluate | 34.33 | 30.82 | 89.8 |

As is apparent from the results set forth in Table 6, polishing with OR-6 adsorbent reduced the overall mass of the solid liquid extraction filtrate by 6.48 grams, while maintaining the majority of the THC mass. As a result, the overall purity of the THC increased from 76.2 wt. % to 89.8 wt. %.

### Example 6—Polishing Using OR-5 Adsorbent

A solid liquid extraction filtrate was solubilized in an ethanolic (90:10 ethanol to water) mixture (51.8 mg/mL) and purified using a lab scale preparation chromatography setup. The lab scale preparation chromatography setup contained one chromatographic column, 10 mm in diameter and 250 mm in length, equipped with OR-5 adsorbent. The chromatographic column was connected to a single pump with a flow rate of 2.0 mL/min and an ethanolic mixture as the mobile phase. The purification process was maintained at a temperature of 25° C., and the solvent was removed from the resulting eluate. The results of the purification process are set forth in Table 7. In addition, the column equipped with OR-5 adsorbent was flushed with ethanol to recover any residual THC that had adsorbed to the column. The OR-5 adsorbent was regenerated using an acetone wash.

TABLE 7
Effect on Polishing with OR-6 Adsorbent

| | Total Mass | THC Mass | THC Purity |
|---|---|---|---|
| Stream | (g) | (g) | (wt. %) |
| Solid liquid extraction | 2.33 | 1.78 | 76.4 |
| filtrate | | | |
| OR-5 eluate | 1.87 | 1.60 | 85.6 |

As is apparent from the results set forth in Table 7, polishing with OR-5 adsorbent reduced the overall mass of the solid liquid extraction filtrate by 0.46 grams, while maintaining the majority of the THC mass. As a result, the overall purity of the THC increased from 76.4 wt. % to 85.6 wt. %.

### Example 7—Polishing Using OR-10 Adsorbent

A solid liquid extraction filtrate was solubilized in an ethanolic (85:15 ethanol to water) mixture (25 mg/mL) and purified using a commercial scale preparation chromatography setup. The commercial scale preparation chromatography setup contained one chromatographic column, 18 inches in diameter and 60 inches in length, equipped with OR-10 adsorbent. The chromatographic column was connected to a single pump with a flow rate of 1.5-2 L/min and an ethanolic mixture as the mobile phase. The purification process was maintained at a temperature of 25° C., and the solvent was removed from the resulting eluate. The results of the purification process are set forth in Table 8.

TABLE 8
Effect on Polishing with OR-10 Adsorbent

| | Total Mass | THC Mass | THC Purity |
|---|---|---|---|
| Stream | (g) | (g) | (wt. %) |
| Solid liquid extraction | 257.5 | 195.7 | 76 |
| filtrate | | | |
| OR-10 eluate | 207.2 | 176.1 | 85 |

As is apparent from the results set forth in Table 8, polishing with OR-10 adsorbent reduced the overall mass of the solid liquid extraction filtrate by 50.3 grams, while maintaining the majority of the THC mass. As a result, the overall purity of the THC increased from 76 wt. % to 85 wt. %.

### Example 8—Decolorization Using OR-6 Adsorbent and Polar Solvent

Crude cannabis extract was decarboxylated at a temperature of 100° C. to 120° C. and a pressure of −0.6 atm to −0.74 atm for a period of 5 to 10 hours to produce 59.10 g of decarboxylated crude cannabis extract. The decarboxylated crude cannabis extract was solubilized in an ethanolic mixture (63 mg/mL) and purified using a lab scale preparation chromatography setup. The lab scale preparation chromatography setup contained one chromatographic columns, 22 mm in diameter and 300 mm in length, equipped with OR-6 adsorbent. The chromatographic column was connected to a single pump with a flow rate of 5 mL/min and an ethanolic mixture as the mobile phase. The purification process was maintained at a temperature of 25° C., and the solvent was removed from the resulting eluate. The results of the purification process are set forth in Table 9.

TABLE 9
Effect on Decolorization with OR-6 Adsorbent and Polar Solvent

| | Total Mass | THC Mass | THC Purity |
|---|---|---|---|
| Stream | (g) | (g) | (wt. %) |
| Decarboxylated crude | 59.10 | 28.37 | 48 |
| cannabis extract | | | |
| OR-6 eluate | 48.07 | 25.50 | 53 |

As is apparent from the results set forth in Table 9, purification with OR-6 adsorbent reduced the overall mass of the decarboxylated crude cannabis extract by 11.03 grams, while maintaining the majority of the THC mass. As a result, the overall purity of the THC increased from 48 wt. % to 53 wt. %.

### Example 9—Polishing Using OR-5 Adsorbent

The OR-6 eluate stream from Example 8 was solubilized in an ethanolic mixture (26.8 mg/mL) and purified using a lab scale preparation chromatography setup. The lab scale preparation chromatography setup contained one chromatographic column, 22 mm in diameter and 300 mm in length, equipped with OR-5 adsorbent. The chromatographic column was connected to a single pump with a flow rate of 5.0 mL/min and an ethanolic mixture as the mobile phase. The purification process was maintained at a temperature of 25° C., and the solvent was removed from the resulting eluate. The results of the purification process are set forth in Table 10. In addition, the column equipped with OR-5 adsorbent was flushed with ethanol to recover any residual THC that had adsorbed to the column. The OR-5 adsorbent was regenerated using an acetone wash.

TABLE 10
Polishing Using OR-5 Adsorbent

| | Total Mass | THC Mass | THC Purity |
|---|---|---|---|
| Stream | (g) | (g) | (wt. %) |
| OR-6 eluate | 1.40 | 0.73 | 52 |
| OR-5 eluate | 1.06 | 0.69 | 65 |

As is apparent from the results set forth in Table 10, polishing with OR-5 adsorbent reduced the overall mass of the OR-6 eluate by 0.34 grams, while maintaining the majority of the THC mass. As a result, the overall purity of the THC increased from 52 wt. % to 65 wt. %. Example 10—Pigments and Carbohydrate Removal Using OR-3 Adsorbent

The OR-5 eluate from Example 9 was solubilized in heptanes (20.2 mg/mL) and purified using a lab scale preparation chromatography setup. The lab scale preparation chromatography setup contained one chromatographic cartridge with 6 mL volume containing 2 grams of OR-3 adsorbent. The chromatographic cartridge was connected to a single pump with a flow rate of 0.5 mL/min and heptanes as the mobile phase. The purification process was maintained at a temperature of 25° C., and the solvent was removed from the resulting eluate. The results of the purification process are set forth in Table 11.

TABLE 11
Effect on Pigments and Carbohydrate
Removal with OR-3 Adsorbent

| | Total Mass | THC Mass | THC Purity |
|---|---|---|---|
| Stream | (g) | (g) | (wt. %) |
| OR-5 eluate | 0.74 | 0.48 | 64.9 |
| OR-3 eluate | 0.43 | 0.39 | 90.7 |

As is apparent from the results set forth in Table 11, purification with OR-3 adsorbent reduces the overall mass of the OR-5 eluate by 0.31 grams, while maintaining the majority of the THC mass. As a result, the overall purity of the THC increased from 64.9 wt. % to 90.7 wt. %.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims, while the description, abstract and drawings are not to be used to limit the scope of the invention. The invention is specifically intended to be as broad as the claims below and their equivalents.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. As used herein, the term “exemplary” indicates an example thereof and does not suggest a best or optimal of the recited item. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of purifying tetrahydrocannabinol (THC) from a composition containing THC and at least one impurity, the method comprising:
passing a feedstock stream comprising the composition through one or more stationary phases to provide an eluate stream having a higher purity of the THC than in the feedstock stream as measured by weight percentage of the THC content,
the one or more stationary phases comprising:
a first adsorbent comprising a silica adsorbent having Si—OH groups and an average particle diameter between 60-200 microns;
a second adsorbent comprising a modified hydrophobic adsorbent having an average bulk density of from about 0.4 g/mL to about 0.6 g/mL, the modified hydrophobic adsorbent comprising at least one of a styrene-divinylbenzene (DVB) resin or a poly(methyl methacrylate) (PMMA) resin;
a third adsorbent comprising a modified activated carbon adsorbent having an average particle size range of from about 40 to about 1700 microns;
or a mixture thereof.

2. The method of claim 1, wherein said at least one impurity comprises at least one of pesticides, waxes, lipids, pigments, sugars, carbohydrates, proteins, chlorophyll, and mixtures thereof.

3. The method of claim 1, wherein said at least one impurity comprises a second cannabinoid selected from cannabidiol (CBD), cannabidiolic Acid (CBDA), cannabigerol (CBG), cannabinol (CBN), tetrahydrocannabinolic acid (THCA), and combinations thereof.

4. The method of claim 1, further comprising:
passing a feedstock stream comprising the composition through one stationary phase disposed in a single column to provide an eluate stream having a higher purity of the THC than in the feedstock stream as measured by weight percentage of the THC content.

5. The method of claim 1, further comprising:
passing a feedstock stream comprising the composition through one stationary phase disposed in at least two columns to provide an eluate stream having a higher purity of the THC than in the feedstock stream as measured by weight percentage of the THC content.

6. The method of claim 1, further comprising:
passing a feedstock stream comprising the composition through more than one stationary phase disposed in at least two columns to provide an eluate stream having a higher purity of the THC than in the feedstock stream as measured by weight percentage of the THC content.

7. The method of claim 5, wherein at least two columns of said at least two columns are arranged in a SMB configuration to form a SMB zone, and wherein passing a feedstock stream comprising the composition through one stationary phase comprises passing the feedstock stream through the SMB zone.

8. The method of claim 4, wherein the feedstock stream, the stationary phase, and the eluate comprise a first feedstock stream, a first stationary phase, and a first eluate, respectively, the method further comprising:
passing the first eluate through a second stationary phase to form a second eluate having a higher purity of THC than in the first eluate as measured by weight percentage of the THC content.

9. The method of claim 8, further comprising:
passing the second eluate through a third stationary phase to form a third eluate having a higher purity of THC than in the second eluate as measured by weight percentage of the THC content.

10. The method of claim 4, wherein the feedstock stream, the stationary phase, and the eluate comprise a first feedstock stream, a first stationary phase, and a first eluate, respectively, the method further comprising:
passing the first eluate through a second stationary phase to form a second eluate having a higher purity of THC than in the first eluate as measured by weight percentage of the THC content;
passing a second feedstock stream through the second stationary phase to form a third eluate having a higher purity of the cannabinoid than in the second feedstock stream as measured by weight percentage of the THC content; and
passing the third eluate through a third stationary phase to form a fourth eluate having a higher purity of the cannabinoid than in the third eluate as measured by weight percentage of the THC content.

11. The method of claim 1, wherein the feedstock stream further comprises a solvent selected from water, ethanol, acetone, ethyl acetate, acetonitrile, pentanes, hexanes, heptanes, methanol, propanol, and a combination thereof.

12. A method of purifying tetrahydrocannabinol (THC) from a composition containing THC and at least one impurity, the method comprising:
passing a first feedstock stream comprising the composition through a first stationary phase to provide a first eluate stream having a higher purity of the THC than in the first feedstock stream as measured by weight percentage of the THC content, the first feedstock stream further comprising a first major solvent of a first polarity;
removing at least some of the first major solvent of the first polarity from the first eluate stream to produce a reduced first eluate stream;
adding a second major solvent of a second polarity to the reduced first eluate stream to produce a second feedstock stream; and
passing the second feedstock stream through a second stationary phase to provide a second eluate stream having a higher purity of the THC than in the second feedstock stream as measured by weight percentage of the THC content;
wherein the first polarity and the second polarity are opposite.

13. The method of claim 12, wherein said at least one impurity comprises at least one of pesticides, waxes, lipids, pigments, sugars, carbohydrates, proteins, chlorophyll, and mixtures thereof.

14. The method of claim 12, wherein said at least one impurity comprises a second cannabinoid selected from cannabidiol (CBD), cannabidiolic Acid (CBDA), cannabigerol (CBG), cannabinol (CBN), tetrahydrocannabinolic acid (THCA), and combinations thereof.

15. The method of claim 12, wherein the first stationary phase and the second stationary phase are each independently selected from:
a first adsorbent comprising a silica adsorbent having Si—OH groups and an average particle diameter between 60-200 microns;
a second adsorbent comprising a modified hydrophobic adsorbent having an average bulk density of from about 0.4 g/mL to about 0.6 g/mL, the modified hydrophobic adsorbent comprising at least one of a styrene-divinylbenzene (DVB) resin or a poly(methyl methacrylate) (PMMA) resin;
a third adsorbent comprising a modified activated carbon adsorbent having an average particle size range of from about 40 to about 1700 microns;
or a mixture thereof.

16. The method of claim 15, wherein the first adsorbent further comprises an average surface area of from about 450 m²/g to about 550 m²/g, and an average pore volume of from about 0.7 mL/g to about 0.9 mL/g.

17. The method of claim 15, wherein the second adsorbent further comprises an average particle diameter of from about 25 microns to about 300 microns, an average surface area of from about 450 m²/g to about 550 m²/g, and an average pore volume of from about 0.7 mL/g to about 0.9 mL/g.

18. The method of claim 15, wherein the third adsorbent further comprises an iodine number greater than about 900.

19. The method of claim 12, wherein the first major solvent is selected from ethanol or water and the second major solvent is selected from hexanes or heptanes.

20. The method of claim 12, wherein the first major solvent is selected from hexanes or heptanes and the second major solvent is selected from ethanol or water.
